(19) United States
(12) Patent Application Publication
(10) Pub. No.: US 2006/0111436 A1
(43) Pub. Date: May 25, 2006

(54) Compositions and treatments for modulating kinase and/or HMG-CoA reductase

(76) Inventor: John Griffin, Atherton, CA (US)

Correspondence Address: WILSON SONSINI GOODRICH & ROSATI, 650 PAGE MILL ROAD, PALO ALTO, CA, 94304-1050, US

(21) Appl. No.: 11/118,098

(22) Filed: Apr. 29, 2005

Related U.S. Application Data
(60) Provisional application No. 60/630,683, filed on Nov. 23, 2004.

(51) Int. Cl. A61K 31/366

(52) U.S. Cl. 514/460

(57) ABSTRACT

The present invention provides compositions of matter, kits and methods for their use in the treatment of kinase-related conditions and/or HMG-CoA reductase-related conditions. In particular, the invention provides compositions for treating immuno-compromised and/or cardiovascular conditions in an animal subject by modulating one or more MAP kinase(s) and/or HMG-CoA reductase, as well as providing formulations and modes of administering such compositions. The invention further provides methods for the rational design of modulators of MAP kinases, HMG-CoA reductase, or both for use in the practice of the present invention.

### RELATED APPLICATIONS

This application claims priority to U.S. Provisional Patent Application Ser. No. 60/630,683, filed Nov. 23, 2004, which is incorporated by reference herein, for all purposes. Related U.S. Provisional Patent Application Ser. No. 60/567,118, filed Apr. 29, 2004, and 60/630,684, filed Nov. 23, 2004, are also incorporated by reference herein, for all purposes.

### BACKGROUND

The pro-inflammatory cytokines, such as tumor necrosis factor-α (TNF-α) and interleukin-1β (IL-1β) contribute to the pathogenesis of various allergic, inflammatory and autoimmune diseases. Conversely, reduced amounts of such cytokines contribute to immuncompromised status, e.g., as may accompany HIV or Hepatitis C infection. As such, multiple therapeutic approaches have been aimed at modulating the expression and/or activity of such pro-inflammatory cytokines. The p38 mitogen activated protein kinases (p38 MAP kinases) play central roles in these signal transduction pathways, providing targets for such therapeutic approaches. However, there remains a need for small molecule compounds capable of activating p38 MAP kinases. Such compounds and compositions can form the basis for pharmaceutical compositions useful in the prevention and treatment of immunocompromised conditions in humans and other mammals.

### BRIEF SUMMARY OF THE INVENTION

Some aspects of the instant invention provide a method of activating a MAP kinase comprising administering an effective amount of a composition comprising a statin lactone. In some embodiments, the activation occurs in a cell other than a brain cell. In some embodiments, the activating occurs by direct activation. In some embodiments, the activating does not occur via a growth factor. In some embodiments, the activating is not reversed by addition of at least one compound selected from farnesyl pyrophosphate, geranylgeranyl pyrophosphate and mevalonte. In some embodiments, the activating is not reversed by addition of a downstream product of mevalonate.

In some embodiments, the MAP kinase is a p38 MAP kinase. In some such embodiments, the MAP kinase is a p38α MAP kinase. In some such embodiments, the statin lactone is simvastatin lactone. In some embodiments, the statin lactone is cerivastatin lactone. In some embodiments, the statin lactone is fluvastatin lactone. In some embodiments, the statin lactone is lovastatin lactone. In some embodiments, the statin lactone is mevastatin lactone. In some embodiments, the statin lactone is not atorvastatin lactone, rosuvastatin lactone, nor pitavastatin lactone.

In some embodiments, the MAP kinase is a p38β MAP kinase. In some such embodiments, the statin lactone is simvastatin lactone. In some embodiments, the statin lactone is cerivastatin lactone. In some embodiments, the statin lactone is fluvastatin lactone. In some embodiments, the statin lactone is atorvastatin lactone. In some embodiments, the statin lactone is not rosuvastatin lactone.

In some embodiments, the MAP kinase is a p387γ MAP kinase. In some such embodiments, the statin lactone is simvastatin lactone. In some embodiments, the statin lactone is cerivastatin lactone. In some embodiments, the statin lactone is rosuvastatin lactone. In some embodiments, the statin lactone is atorvastatin lactone. In some embodiments, the statin lactone is pitavastatin lactone. In some embodiments, the stain lactone is not fluvastatin lactone.

In some embodiments, the MAP kinase is a p38δ MAP kinase. In some such embodiments, the statin lactone is simvastatin lactone. In some embodiments, the statin lactone is cerivastatin lactone. In some embodiments, the statin lactone is rosuvastatin lactone. In some embodiments, the statin lactone is atorvastatin lactone. In some embodiments, the statin lactone is fluvastatin lactone.

In some embodiments, the composition activates at least two MAP kinases. In some embodiments, the at least two MAP kinases are selected from a p38α MAP kinase, a p38β MAP kinase, a p38γ MAP kinase, a p38δ MAP kinase, and a p42 MAP kinase. In some embodiments, the statin lactone is at least one lactone selected from simvastatin lactone, cerivastatin, fluvastatin lactone, rosuvastatin lactone, and atorvastatin lactone. In some embodiments, the MAP kinase is a JNK. In some such embodiments, the activating facilitates a Fas apoptotic pathway.

### BRIEF DESCRIPTION OF THE FIGURES

FIG. 1 illustrates some of the pathways involved in inflammatory signaling cascades and the activation of certain of these pathways by a MAP kinase activator.

FIG. 2 illustrates some of the pathways involved in cholesterol biosynthesis and some of the atherogenic mechanisms of hypercholesterolemia, as well as the interruption of certain of these pathways by an HMG-CoA reductase inhibitor.

FIG. 3 illustrates an example of each of nine classes (a-i) of statin inhibitors of HMG-CoA reductase in lactone form.

### DETAILED DESCRIPTION OF THE INVENTION

I. Kinase and/or HMG-CoA Reductase Modulators

One aspect of the present invention relates to compositions that modulate kinases. In some embodiments, compositions are provided that activate protein kinases, e.g., protein kinases involved in signaling cascades, such as mitogen-activated protein kinases (MAP kinases, MAPKs). These compositions can exert pro-immune and pro-inflammatory effects in vitro and in vivo. In certain embodiments, these compositions can modulate one or more of the types or isoforms of p38 MAP kinase, such as p38α MAPK, p38β MAPK, p38γ MAPK and/or p38δ. In some embodiments, these compositions can modulate stress-activated protein kinases/Jun N-terminal kinases (SAPKs/JNKs).

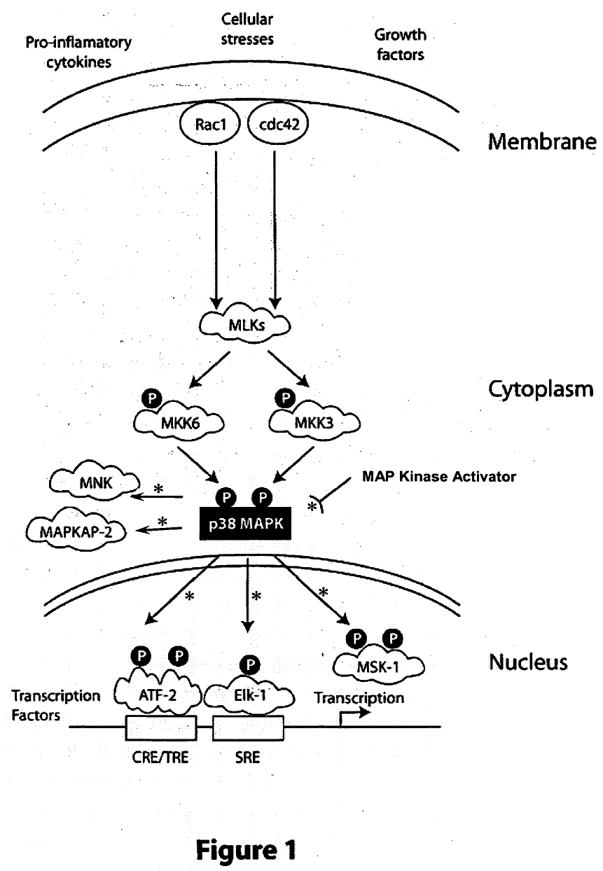

FIG. 1 illustrates some of the pathways involved in inflammatory signaling cascades and the activation of certain of these pathways by a MAP kinase activator. This figure provides an overview only, and is in no way intended to be limiting with respect to the present invention. For example, those skilled in the art will readily appreciate variations and modifications of the scheme illustrated.

As FIG. 1 illustrates, pro-inflammatory cytokines (e.g., TNF-α, and IL-1), as well as cellular/environmental stresses and growth factors, initiate a signal transduction cascade leading to the activation of several serine/threonine kinases, including MKK3, MKK6 and p38 MAP kinase. Chakravarty et al., Ann. Rep. Med. Chem. 37, 177-186 (2002). As is known in the art, the p38 MAP kinases are soluble, intracellular protein serine/threonine kinases which play central roles in signal transduction pathways. Four isoforms of p38 MAPKs are recognized. p38 MAP kinases exist in at least four isoforms, p38α (expressed in all tissues), p38β (expressed in all tissues), p38γ (primarily expressed in skeletal tissue), and p38δ (primarily expressed in the lungs, kidneys, testes, pancreas and small intestine). These enzymes share a Thr-Gly-Tyr dual phosphorylation motif for activation, along with highly conserved amino acid sequences, particularly in the binding pocket for ATP. p38α MAP kinase serves as the primary MAP kinase associated with the pro-inflammatory cytokines of immune and/or inflammatory signally pathways and is phosphorylated on Thr-180 and Tyr-182. See, e.g., Chakravarty et al., supra, (2002).

As FIG. 1 illustrates, activation of p38 MAP kinase by upstream kinases leads to phosphorylation of downstream substrates, including MNK and MAPKAP-2, as well as transcription factors ATF-2, Elk-1, and MSK-1. These in turn control transcription and production, of pro-inflammatory cytokines. FIG. 1 also illustrates points of action of an activator that can increase downstream effects of p38 MAP kinase, illustrated by asterisks. For example, activation of p38α MAP kinase using a compound according to the present invention can increase phosphorylation of p38α MAP kinase, MNK, MAPKAP-2, ATF-2, Elk-1 and/or MSK-1, increasing production of pro-inflammatory cytokines, in certain embodiments, as discussed in detail below.

“Activation” and its grammatical conjugations, such as “activating,” refer to an increase in kinase enzyme activity. Such activation is preferably by at least about 20%, by at least about 50%, by at least about 80%, by at least about 100%, by at least abut 150%, by at least about 200%, by at least about 400%, or by at least about 600% of the activity of the enzyme in the absence of the activating effect, e.g., in the absence of an activator. Conversely, the phrase “does not activate” and its grammatical conjugations can refer to situations where there is less than about 20%, less than about 10%, and preferably less than about 5% increase in enzyme activity in the presence of the compound. Further the phrase “does not substantially activate” and its grammatical conjugations can refer to situations where there is less than about 30%, less than about 20%, and preferably less than about 10% increase in enzyme activity in the presence of the compound.

In preferred embodiments, kinase activation occurs by direct activation. “Direct activation,” and its grammatical conjugations, can refer to stimulating, promoting, increasing, improving, inducing, and/or enhancing a catalytic activity of at least one kinase at least partly through a direct physical interaction between the kinase activator and the kinase. For example, a compound of the present invention can directly activate a p38 MAP kinase by binding, complexing and/or interacting with the kinase at one or more sites of the kinase macromolecule. “Direct activation” does not exclude activation partly involving indirect activation. For example, direct activation can include activation at least partly brought about by direct physical interaction between the activator and the kinase macromolecule, e.g., where at least about 30%, at least about 50%, at least about 80%, or at least about 90% of the increase in enzyme activity is a consequence of direct activation.

“Directly activate” can distinguish from indirect mechanisms, for example, mechanisms by which cellular and/or environmental stresses, growth factors, cytokines and/or other small molecules can activate a protein kinase or MAP kinase signal transduction pathway without a direct physical interaction between the activator and the kinase molecule. For example, the cytokine tumor necrosis factor alpha (TNF α) can activate a MAP kinase signal transduction pathway through a cascade of events, e.g., beginning with the binding of TNF α to cognate receptors located on the exterior surface of cells. Other indirect activators act on MAP kinases via a growth factor, e.g., VEGF and/or bFGF, and/or via promotion of endothelial progenitor cell, neuronal progenitor and/or stem cell migration and/or differentiation. See Chop (WO 03/086379). As another example, the small molecule protein synthesis inhibitor anisomycin broadly activates MAP kinases through a mechanism that has not been characterized as direct interaction between anisomycin and the kinase macromolecule. Thus, it is especially surprising to discover direct activation of MAP kinases by compounds described herein, including statin lactones.

“Direct activation,” and its grammatical conjugations, can also refer to stimulating, promoting, increasing, improving, inducing, and/or enhancing a catalytic activity of at least one kinase by a mechanism that is not reversed by addition of at least one compound selected from farnesyl pyrophosphate, geranylgeranyl pyrophosphate, mevalonte and any other downstream product of mevalonate. In contrast, for example, the carboxylate salt form of simvastatin can indirectly activate protein kinase B (Akt) in endothelial cells through a mechanism apparently involving one or more points “upstream” of Akt in signal transduction pathways, e.g., affected by HMG-CoA reductase inhibition. See, e.g., Kureishi, et al., Nature Medicine 6, 1004-1010 (2000).

In some preferred embodiments, modulation of kinases involves activation of one or more types of kinases accompanied by inhibition of one or more other types of kinases. For example, a compound used in some embodiments of the present invention may inhibit a p38α MAP kinase and activate a non-p38α MAP kinase, preferably by direct activation, as described in more detail above.

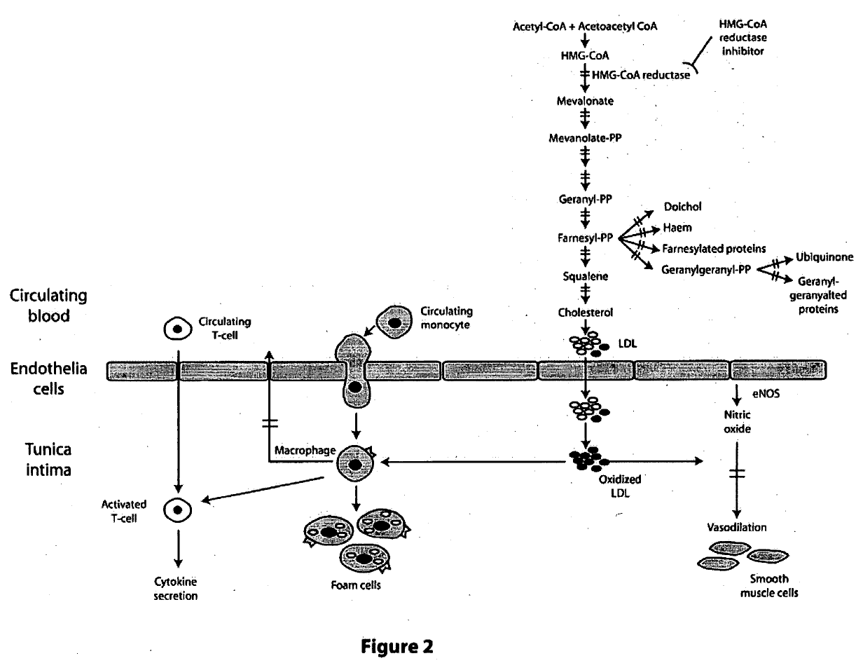

A second aspect of the present invention relates to compositions that modulate, e.g., inhibit, the enzyme 3-hydroxy-3-methyl glutaryl-coenzyme A reductase (HMG-CoA reductase). These compositions can lower cholesterol levels in vitro and in vivo. FIG. 2 illustrates some of the pathways involved in cholesterol biosynthesis and some of the atherogenic mechanisms of hypercholesteremia, as well as the interruption of certain of these pathways by an HMG-CoA reductase inhibitor. This figure provides an overview only, and is in no way intended to be limiting. For example, those skilled in the art will readily appreciate variations and modifications of the scheme illustrated, and more detailed descriptions can be found in standard texts on biochemistry, metabolism, pathophysiology, and the like.

As is known in the art, HMG-CoA reductase catalyzes the committed, rate-limiting step of terpene and cholesterol synthesis in mammalian cells. It thus represents a target for small molecule therapeutics (e.g., the “statins”) aimed at reducing atherogenesis and its associated cardiovascular risks. HMG-CoA reductase acts on 3-hydroxy-3-methyl-glutaryl CoA (HMG-CoA) to produce mevalonate. The pathway also produces other non-sterol isoprenoid products, such as farnesol, dolichol, and ubiquinone. Mevalonate is converted into cholesterol, which is carried mainly in the blood in two specialized particles known as low-density lipoprotein (LDL) and high-density lipoprotein (HDL).

As illustrated in FIG. 2, LDL adheres to the arterial wall and is progressively oxidized. Palinski et al., J. Am. Soc. Nephrol., 13: 1673-1681 (2002). Extensively oxidized LDL is taken up by macrophages to form foam cells, a key feature of atherosclerosis. This leads to recruitment of monocytes and T-cells and secretion of cytokines in immune response cascades. The double bars indicate currently known effects of HMG-CoA reductase inhibitors (e.g., statins) on these processes, not only in reducing the production of cholesterol, but also in modulating immune responses through the actions of other metabolites such as farnesyl pyrophosphate and geranylgeranyl pyrophosphate. For example, geranylgeranyl-PP decreases endothelial cell nitric oxide synthase (eNOS) expression, inhibiting nitric oxide-induced vasodilation. Inhibition of HMG CoA reductase using a compound in accordance with the present invention can also produce these effects, in certain embodiments, as discussed in detail below.

A third aspect of this invention relates to compositions that modulate both kinase and HMG-CoA reductase activities. Such compositions can activate one or more other types of p38 kinase and inhibit HMG CoA reductase as well as one or more types of p38 MAP kinase. In some embodiments, such compositions can stimulate production of HDL while inhibiting both cholesterol biosynthetic pathways and inflammatory responses in vitro, and can exert, for example, lipid-modulating, anti-inflammatory, and anti-atherogenic properties in vivo. Further, such compositions can provide superior benefits in treating HMG-CoA reductase-related conditions, such as cardiovascular disease, compared with treatments that modulate HMG-CoA reductase but not a MAP kinase, due to the interplay between inflammatory and cardiovascular disorders. In other embodiments, such compositions can provide superior benefits in treating kinase-related conditions, such as immunocompromised conditions, compared with treatments that modulate MAP kinase but not HMG-CoA reductase, again due to the interplay between inflammatory and cardiovascular conditions.

In certain embodiments, the compositions of the present invention comprise compounds of formulas I and/or II, as illustrated below, 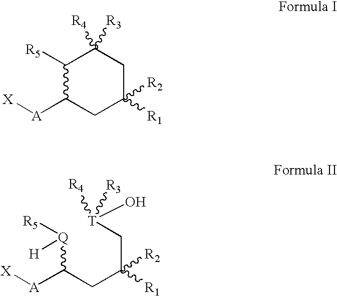
wherein A is a covalent bond, methylene, 1,2-oxamethylene, 1,2 ethylene, 1,2-ethynylene, 1,2 ethenylene, 1,3 propylene or 1,3 propenylene, preferably 1,2-ethylene or E-1,2-ethenylene; X comprises a lipophilic moiety; Q is preferably oxygen, sulfur or nitrogen; T is preferably carbon or sulfur; R₁ is hydroxy, lower alkoxy, hydrogen or lower alkyl, preferably hydroxy; R₂ is hydrogen or lower alkyl, preferably hydrogen; R₃ and R₄ are preferably hydrogen, oxygen or together an oxygen atom; and R₅ is preferably hydrogen, lower alkyl, substituted lower alkyl, aralkyl, substituted aralkyl, heteroaralkyl, or substituted heteroaralkyl, wherein the compound is not a known statin lactone.

In some embodiments, T is carbon and R₃ and R₄ are preferably hydrogen or are preferably together an oxygen atom. In some embodiments, T is sulfur R₃ and R₄ are preferably oxygen atoms. In some embodiments, Q is nitrogen, R₅ is hydrogen, lower alkyl, substituted lower alkyl, aralkyl, substituted aralkyl, heteroalralkyl, or substituted heteroaralkyl.

In some embodiments, the lipophilic moiety X comprises an aromatic ring. As used herein, a lipophilic moiety can refer to a molecular entity or a portion thereof, having a tendency to dissolve in fat-like solvents, e.g., in a hydrocarbon solvent. Such moieties can also be referred to as hydrophobic moieties. In some embodiments, the lipophilic moiety X comprises at least one lipophilic moiety selected from an alicyclic moiety, a carbocyclic aromatic moiety, and a heterocyclic aromatic moiety.

In some embodiments, the compounds of Formula I and II are novel analogs of known inhibitors of HMG-CoA reductase, e.g., statin drugs. A “statin” as used herein can refer to any compound that can inhibit HMG-CoA reductase, generally comprising formula I or II. Statins include, e.g., without being limited to, mevastatin, lovastatin, simvastatin, pravastatin, fluvastatin, atorvastatin, cerivastatin, rosuvastatin, pitavastatin, glenvastatin, bervastatin, dalvastatin, eptastatin, dihydroeptastatin, itavastatin, L-154819, advicor, L-654969, and other statin drugs used to treat hypercholesterolemia and other lipid disorders. For example, the lipophilic moiety X may comprise a lipophilic moiety of a statin analog. The statin analog may be an analog of a natural statin, e.g., an analog of simvastatin, or an analog of a synthetic statin, e.g., an analog comprising at least one moiety selected from a lactone moiety, a lactam moiety, a thiolactone moiety, a cyclic sulfonic ester moiety, a cyclic sulfuric ester moiety, a cyclic sulfonic amide moiety, a cyclic sulfuric amide moiety, a tetrahydropyran moiety, and a tetrahydrothiopyran moiety. Statins are classified in the art as natural or synthetic statins depending on their origin. Natural stains include, for example, mevastatin, lovastatin, simvastatin, pravastatin, and the like. Synthetic statins include, for example, atorvastatin, fluvastatin, cerivastatin, rosuvastatin, pitavastatin, and glenvastatin.

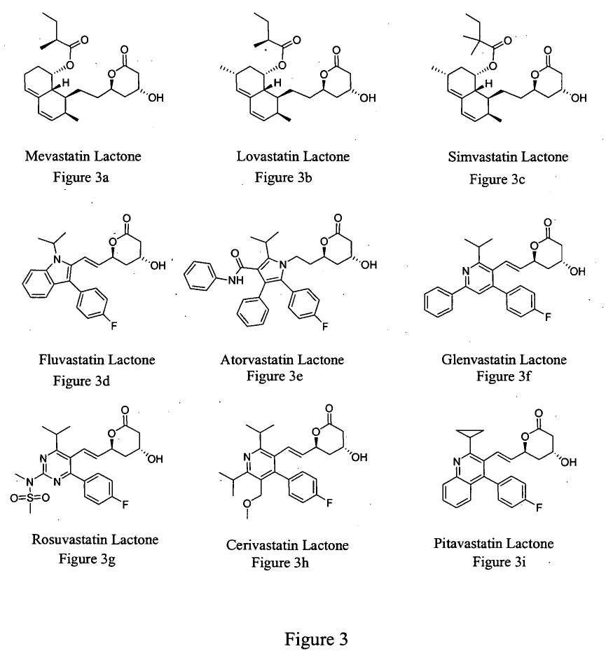

FIG. 3 illustrates an example of each of nine known classes (a-i) of statin inhibitors of HMG-CoA reductase in the lactone form of formula. FIG. 3a illustrates mevastatin lactone, derivatives of which are preferred in certain embodiments of the invention. FIG. 3b illustrates lovastatin lactone, derivatives of which are preferred in certain embodiments of the invention. FIG. 3c illustrates simvastatin lactone, derivatives of which are preferred in certain preferred embodiments. FIG. 3d illustrates fluvastatin lactone, derivatives of which are preferred in certain embodiments of the invention. FIG. 3e illustrates atorvastatin lactone, derivatives of which are preferred in certain embodiments of the invention. FIG. 3f illustrates glenvastatin lactone, derivatives of which are preferred in certain embodiments of the invention. FIG. 3g illustrates rosuvastatin lactone, derivatives of which are preferred in certain embodiments of the invention. FIG. 3h illustrates cerivastatin lactone, derivatives of which are preferred in certain embodiments of the invention FIG. 3i illustrates pitavastatin lactone, derivatives of which are preferred in certain embodiments of the invention. Each structure in FIG. 3 also illustrates an absolute stereochemistry of a 3-hydroxy-δ-lactone structure with respect to its two stereogenic centers, which is preferred in some embodiments. Also included are MAP kinase modulators with inhibitory activity. (See, e.g., U.S. Provisional Application No. 60/567,118, filed Apr. 29, 2004, entitled “Compositions and Treatments for Inflammatory Conditions,” incorporated herein by reference).

In some embodiments, compounds of the present invention can inhibit hydroxymethyl-glutaryl-CoA reductase in the form shown in formula II. Formula II represents a ring opened, hydrolyzed form of the compound of formula I and is meant to include salt forms thereof. Those of skill in the art will recognize that a compound of formula I can open to the acid form with (reversible) addition of water, and may further equilibriate, e.g., to the deprotonated (salt) form with the loss of a proton to give the corresponding ion. For example, the dihydroxy-carboxylate side chain of statins can be induced to undergo a cyclodehydration to form a 3-hydroxy-δ-lactone structure of Formula I wherein A is 1,2-ethylene or E 1,2-ethenylene, Q is oxygen, T is carbon, R₁ is hydroxy, R₂ is hydrogen and R₃ and R₄ are together a carbonyl oxygen atom. It further will be recognized by those in the art that a rapid equilibrium exists between the protonated and deprotonated forms, and that the deprotonated form usually predominates at neutral and basic pH. Reference to “formula II” or “II” herein refers to both protonated and deprotonated forms. Moreover, the present invention encompasses both the protonated and deprotonated (i.e., salt) forms of the compounds disclosed herein.

Further, those of skill in the art will recognize that certain compounds of the present invention may exhibit the phenomena of tautomerism, conformational, isomerism, geometric isomerism and/or optical isomerism. It should be understood that the invention encompasses any tautomeric, conformational isomeric, optical isomeric and/or geometric isomeric forms of the kinase and/or HMG-CoA reductase modulators described herein, as well as mixtures of these various different forms. For example, optically active modulators of the present invention may be administered in enantiomerically pure (or substantially pure) form or as a mixture of detrorotatory and levorotatory enantiomers, such as in a racemic mixture. It can also be appreciated that the compounds disclosed herein can exist in different—crystalline forms, including, e.g., polymorphs. The invention encompasses these different crystalline forms, mixtures of different crystalline forms, and pure or substantially pure crystalline forms.

The compounds disclosed in this invention can be produced by methods known in the art as they are derivatives of classes of compounds known in the art. For example, where the compounds are derivatives of classes of compounds known in the art, they may be synthesized based on appropriate variations of known synthetic procedures. For example, the synthesis of statins is described in Roth et al., J. Med. Chem., 34:357-366 (1991); Krause et al., J. Drug Dev., 3(Suppl. 1):255-257 (1990); and Karanewsky, et al., J. Med. Chem. 33:2952-2956 (1990). Known methods for the synthesis of statin inhibitors of HMG-CoA reductase in the lactone form and in analogous dihydropyran and lactam forms can be adapted to synthesis of compounds of Formula I. Examples are also provided in Examples 1, 2, and 3 provided below. Further, specific examples of the present invention can be made by variations of methods known to those of skill in the art and provided herein, for example, where starting materials, solvents, and other reaction conditions are varied to optimize yields.

In certain embodiments, the compounds of the present invention can be made using commercially available compounds as starting materials. For example, lactones of formula I can be prepared from commercially available salts of HMG-CoA reductase inhibitors. For instance, commercially available calcium or sodium salts of atorvastatin, fluvastatin and rosuvastatin may be converted to their protonated free acid forms by extracting the salt forms from weakly acidic aqueous media into an aprotic organic solvent such as ethyl acetate. By stirring the free acid forms in this or another aprotic organic solvent (such as toluene) approximately at or above room temperature, spontaneous conversion to the lactone form occurs over a timeframe of about hours to about days. The lactone forms may be conveniently purified by any methods known in the art, including by column, preparative thin-layer, rotating, or high-pressure chromatography on silica gel columns using standard eluting solvent systems such as about 5:1 (v:v) acetone:ethyl acetate.

In other embodiments, compounds of the present invention can be made from modifying intermediates of synthesis pathways of known statins. For example, a group can be replaced by reactive groups such as an amino, halogen, or hydroxy group, or a metal derivative such as sodium, magnesium, or lithium, and these groups further reacted. Further, those skilled in art will recognize that compounds of the present invention synthesized by various art-known methods will give cis/trans isomers, E/Z forms, diastereomers, and optical isomers, all of which are included in the present invention.

Another aspect of the present invention relates to analogs of known lipophilic AHMG-CoA reductase inhibitors, e.g. statins, having structures modified to favor and/or enforce a closed ring structure, for example, a ring structure or cyclic form that is not hydrolyzed or not substantially hydrolyzed to its carboxylic acid or carboxylate forms. In some embodiments, for example, the compound may comprise at least one moiety selected from a lactone moiety, a lactam moiety, a thiolactone moiety, a cyclic sulfonic ester moiety, a cyclic sulfuric ester moiety, a cyclic sulfonic amide moiety, a cyclic sulfuric amide moiety, a tetrahydropyran moiety, and a tetrahydrothiopyran moiety. “Not hydrolyzed” and “not substantially hydrolyzed,” along with their grammatical conjugations, include situations where some of the compound is hydrolyzed while some is not hydrolyzed. Preferably, at least about 50%, at least about 75%, at least about 90%, and more preferably at least about 95% of the compound is in a ring structure of cyclic form at equilibrium, in situations where the compound is not substantially hydrolyzed. Preferably, at least about 70%, at least about 80%, at least about 90%, and more preferably at least about 95%, and even more preferably at least about 98% of the compound is in a ring structure or cyclic form at equilibrium, in situations where the compound is not hydrolyzed.

Formulas III and IV illustrate two examples of modified closed ring structures that are analogs of a δ-lactone. Formula III represents a des-oxo-form, where the carbonyl oxygen is removed, thereby preventing or inhibiting hydrolytic ring opening. Formula IV represents a δ-lactam form, where a nitrogen replaces an oxygen in the ring, which increases the hydrolytic stability of the cyclic form.

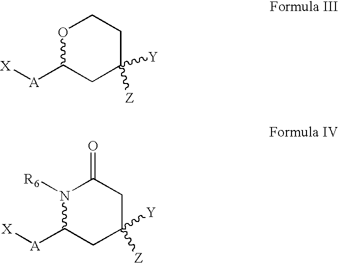

In these formulas, X comprises a lipophilic moiety. In some preferred embodiments, X comprises a lipophilic moiety of a statin, including, for example, the statins of FIG. 3. Preferably, X comprises a lipophilic moiety bearing at least one aromatic substituent, more preferably an aromatic moiety of a synthetic statin. A represents a covalent bond or a substituted or unsubstituted alkylene, alkenylene, or alkynylene linker of 2-6 carbons, optionally containing a heteroatom, such as O, N, or S. A is preferably a covalent bond, methylene, 1,2-oxamethylene, 1,2-ethylene, 1,2-ethynylene, 1,2-ethenylene, 1,3-propylene or 1,3-propenylene. More preferably, A is 1,2-ethylene or E-1,2-ethenylene. Y is hydrogen or a lower alkyl, preferably hydrogen. Z is a hydroxy (—OH) group or hydrogen, preferably a hydroxy group. And R₆ is hydrogen, aryl, substituted aryl, heteroaryl, substituted heteroaryl, alkaryl, substituted alkaryl, benzyl, substituted benzyl, napthylmethylene, or substituted napthlymethylene. Preferably, R₆ is alkaryl or substituted alkaryl; more preferably R₆ is benzyl, substituted benzyl, napthylmethylene, or substituted napthlymethylene. Further, each of the four possible stereoisomers, arising from the two possible absolute configurations at each of the two stereogenic centers of formulas III and IV, are contemplated embodiments of the invention.

The present invention relates to these compounds and compositions therof, to pharmaceutical formulations containing these compounds, and to the use of such compounds and/or their corresponding acids of formula II in treating MAP kinase-related and/or HMG-CoA reductase-related conditions.

II. Methods of Modulating MAP Kinases and/or HMG Co-A Reductase

Another aspect of the present invention relates to using compositions and kits comprising one or more compounds described herein to modulate one or more types of kinases and/or HMG CoA reductase. The present invention envisions use of such compositions and kits to provide different profiles of modulation of types of kinases.

For example, in some embodiments, a compound described herein may modulate the activity of different types of kinases in different ways, e.g., activating one or more types, inhibiting one or more types, and/or having no or substantially no effect on one or more still other types. In still some embodiments, the activation and/or inhibition may occur with different potencies, different absolute efficacies, different maximal and half-maximal concentrations, and the like, with respect to one or more different types of kinases. The varying kinds and degrees of activation and/or inhibition can provide a profile of modulation for a composition described herein.

The ability to reduce enzyme activity is a measure of the inhibitory potency or the activity of a compound towards or against the enzyme. Inhibitory potency is preferably measured by cell free, whole cell and/or in vivo assays in terms of IC50, Kᵢ and/or ED50 values. An IC50 value represents the concentration of a compound required to inhibit enzyme activity by half (50%) under a given set of conditions. A Kᵢ value represents the equilibrium affinity constant for the binding of an inhibiting compound to the enzyme. An ED50 value represents the dose of a compound required to effect a half-maximal response in a biological assay. Further details of these measures will be appreciated by those of ordinary skill in the art, and can be found in standard texts on biochemistry, enzymology, and the like.

The ability to activate enzyme activity is a measure of the inducing potency or the activity of a compound towards or against the enzyme. Inducing potency is preferably measured by cell free, whole cell and/or in vivo assays in terms of AC50 or Max % Act values. An AC50 value represents the concentration of a compound required to activate enzyme activity by 50% under a given set of conditions. A Max % Act value represents the concentration at which a maximum increase in enzyme activity is observed in a biological assay. Further details of these measures will be appreciated by those of ordinary skill in the art, and can be found in standard texts on biochemistry, enzymology, and the like.

Some embodiments provide a method of activating a kinase comprising administering an effective amount of a compound of formula I, provided above. Preferably, the activation involves direct activation. In some embodiments, the kinase is a protein kinase, e.g., a protein kinase B (PKB or Akt). In some embodiments, the kinase is a protein serine-threonine kinase, e.g., a MAP kinase a p38 MAP kinase (including p38α MAP kinase, p38β MAP kinase, p38γ MAP kinase, p38δ MAP kinase). In preferred embodiments, the stereochemistry of the compound is that of the structures illustrated in FIG. 3.

Some embodiments provide methods of activating a kinase comprising administering an effective amount of a compound of formula I, provided above, as well as inhibiting HMG-CoA reductase. In some embodiments, the kinase activated is MAP kinase and activation preferably occurs via direct activation. In some embodiments, HMG-CoA reductase inhibition occurs via a ring-opened, hydrolyzed form of a compound of formula I (e.g., a compound of formula II and/or a salt thereof). In still some embodiments, the lactone used is not substantially hydrolyzed to an acid form and/or does not substantially inhibit HMG-CoA reductase.

Some embodiments provide methods of activating a MAP kinase by administering an effective amount of a composition comprising a known statin lactone. Table I, for example, illustrates profiles of modulation of activities of various MAP kinases by lactone forms of each of five classes of known statin inhibitors of HMG-CoA reductase (i.e., atorvastatin lactone, fluvastatin lactone, simvastatin lactone, rosuvastatin lactone, and pitavastatin). Table I illustrates the effects of each of these compounds against each of four human p38 MAP kinase isoforms observed in cell free assays. Additional details are provided in Examples 5, 6, 7 and 8 below.

Table I summarizes modulation of activities of various p38 MAP kinase isoforms by each of six classes of statin inhibitors of HMG-CoA reductase, as acid salts and as lactones (i.e., atorvastatin, fluvastatin, simvastatin, rosuvastatin, and pitavastatin, as well as cerivastatin).

TABLE I

| | p38α | | | p38β | | |
|---|---|---|---|---|---|---|
| Compound | IC50^{a} | AC50^{b} | Max % Act^{c} | IC50 | AC50 | Max % Act |
| Atorvastatin calcium | >100 μM | ANO^{d} | | NT | NT | |
| Atorvastatin lactone | 31 μM | ANO | | 94 μM | | 19% (10 μM) |
| Fluvastatin sodium | 34 μM | ANO | | NT | NT | |
| Fluvastatin lactone | 48 μM | | 34-41% (30 μM) | ˜50 μM | | 43% (30 μM) |
| Simvastatin sodium | >100 μM | NT^{f} | | NT | NT | |
| Simvastatin lactone | INO^{e} | ˜20 μM | 207% (100 μM) | INO | ˜15 μM | 185% (100 μM) |
| Rosuvastatin sodium | 92 μM | ANO | | NT | NT | |
| Rosuvastatin lactone | >100 μM | ANO | | >100 μM | ANO | |
| Pitavastatin calcium | 101 μM | ANO | | NT | NT | |
| Pitavastatin lactone | >300 μM | ANO | | NT | NT | |
| Cerivastatin calcium | >30 μM | ANO | | NT | NT | |
| Cerivastatin lactone | INO | ˜10 μM | 76% (100 μM) | INO | ˜5 μM | 143% (10 μM) |

| | |
|---|---|
| p38γ | p38δ |

| Compound | IC50 | AC50 | Max % Act | IC50 | AC50 | Max % Act |
|---|---|---|---|---|---|---|
| Atorvastatin calcium | NT | NT | | NT | NT | |
| Atorvastatin lactone | ˜50 μM | ˜10 μM | 62% | 83 μM | 30-100 μM | 112% |
| | | | (10 μM) | | | (30 μM) |
| Fluvastatin sodium | NT | NT | | NT | NT | |
| Fluvastatin lactone | >100 μM | ANO | | ˜90 μM | ˜3 μM | 440% |
| | | | | | | (30 μM) |
| Simvastatin sodium | NT | NT | | NT | NT | |
| Simvastatin lactone | INO | 3-10 μM | 127% | INO | ˜2 μM | 611% |
| | | | (100 μM) | | | (100 μM) |
| Rosuvastatin sodium | NT | NT | | NT | NT | |
| Rosuvastatin lactone | INO | | 26% | INO | ˜50 μM | 138% |
| | | | (100 μM) | | | (100 μM) |
| Pitavastatin calcium | INO | ANO | | NT | NT | |
| Pitavastatin lactone | INO | 3-10 μM | 257% | NT | NT | |
| | | | (100 μM) | | | |
| Cerivastatin calcium | NT | NT | | NT | NT | |
| Cerivastatin lactone | INO | ˜5 μM | 115% | INO | ˜8 μM | 158% |
| | | | (100 μM) | | | (100 μM) |

^{a}Concentration of compound required to inhibit phosphorylation of myelin basic protein by
recombinant human p38 MAP kinase enzymes by 50%.
^{b}Concentration of compound required to activate phosphorylation of myelin basic protein by
recombinant human p38 MAP kinase enzymes by 50%.
^{c}Maximum observed increase in p 38 MAP kinase activity in presence of compound.
Concentration at which maximum increase in activity is observed is provided in parentheses.
^{d}ANO = Activation of p38 MAP kinase not observed at any concentration of compound
tested.
^{e}INO = Inhibition of p38 MAP kinase not observed at any concentration of compound tested.
^{f}NT = Not tested.

As Table I illustrates, in some embodiments, p38α MAP kinase is activated by simvastatin lactone. For example, p38α MAP kinase can be activated by simvastatin lactone administered to a concentration of less than about 10 μM, less than about 20 μM, less than about 40 μM, less than about 60 μM, less than about 80 μM, less than about 100 μM, or less than about 120 μM. Simavastatin lactone may be used to increase enzymatic activity of p38α MAP kinase by at least about 20%, at least about 30%, at least about 50%, at least about 100%, at least about 150%, at least about 200%, or at least about 207%.

In some embodiments, p38α MAP kinase is activated by cerivastatin lactone. For example, p38α MAP kinase can be activated by cerivastatin lactone administered to a concentration of less than about 5 μM, less than about 10 μM, less than about 40 μM, less than about 60 μM, less than about 80 μM, less than about 100 μM, or less than about 120 μM. Simavastatin lactone may be used to increase enzymatic activity of p38α MAP kinase by at least about 20%, at least about 30%, at least about 40%, at least about 50%, at least about 60%, at least about 70%, or at least about 76%.

In some embodiments, p38α MAP kinase is activated by fluvastatin lactone. For example, p38α MAP kinase can be activated by fluvastatin lactone administered to a concentration of less than about 3 μM, less than about 6 μM, less than about 10 μM, less than about 20 μM, less than about 30 μM, less than about 40 μM, or less than about about 45 μM. Fluvastatin lactone may be used to increase enzymatic activity of p38α MAP kinase by at least about 20%, at least about 30%, at least about 34%, at least about 40%, at least about 41%, or at least about 45%. In still some embodiments, p38α MAP kinase is inhibited by fluvastatin lactone. For example, p38α MAP kinase can be inhibited by fluvastatin lactone administered to a concentration of at least about 45 μM, at least about 48 μM, or at least about 50 μM.

In some embodiments, p38α MAP kinase is activated by lovastatin lactone. In some embodiments, p38α MAP kinase is activated by mevastatin lactone. In some embodiments, p38α MAP kinase is not activated by atorvastatin lactone, rosuvastatin lactone, nor pitavastatin lactone.

In some embodiments, p38β MAP kinase is activated by simvastatin lactone. For example, p38β MAP kinase can be activated by simvastatin lactone administered to a concentration of less than about 10 μM, less than about 15 μM, less than about 30 μM, less than about 60 μM, less than about 80 μM, less than about 100 μM, or less than about 120 μM. Simvastatin lactone may be used to increase enzymatic activity of p38β MAP kinase by at least about 20%, at least about 50%, at least about 80%, at least about 100%, at least about 150%, at least about 185%, or at least about 200%.

In some embodiments, p38β MAP kinase is activated by cerivastatin lactone. For example, p38β MAP kinase can be activated by cerivastatin lactone administered to a concentration of less than about 2 μM, less than about 2 μM, less than about 5 μM, less than about 8 μM, less than about 9 μM, less than about 10 μM, or less than about 15 μM. Cerivasatin lactone may be used to increase enzymatic activity of p38β MAP kinase by at least about 60%, at least about 80%, at least about 100%, at least about 120%, at least about 140%, at least about 143%, or at least about 150%.

In some embodiments, p38β MAP kinase is activated by fluvastatin lactone. For example, p38β MAP kinase can be activated by fluvastatin lactone administered to a concentration of less than about 3 μM, less than about 6 μM, less than about 15 μM, less than about 20 μM, less than about 30 μM, less than about 40 μM, or less than about 45 μM. Fluvastatin lactone may be used to increase enzymatic activity of p38β MAP kinase by at least about 10%, at least about 20%, at least about 30%, at least about 40%, at least about 43%, or at least about 50%. In still some embodiments, p38β MAP kinase is inhibited by fluvastatin lactone. For example, p38β MAP kinase can be inhibited by fluvastatin lactone administered to a concentration of at least about 45 μM, at least about 50 μM, or at least about 55 μM.

In some embodiments, p38β MAP kinase is activated by atorvastatin lactone. For example, p38β MAP kinase can be activated by atorvastatin lactone administered to a concentration of less than about 1 μM, less than about 5 μM, less than about 10 μM, or less than about 15 μM. Atorvastatin lactone may be used to increase enzymatic activity of p38β MAP kinase by at least about 10%, at least about 15%, at least about 19%, at least about 20%, or at least about 25%. In still some embodiments, p38β MAP kinase is inhibited by atorvastatin lactone. For example, p38β MAP kinase can be inhibited by atorvastatin lactone administered to a concentration of at least abut 90 μM, at least about 94 μM, or at least about 100 μM. In some embodiments, p38β MAP kinase is not activated by rosuvastatin lactone.

In some embodiments, p38γ MAP kinase is activated by simvastatin lactone. For example, p38γ MAP kinase can be activated by simvastatin lactone administered to a concentration of less than about 3 μM, less than about 10 μM, less than about 30 μM, less than about 50 μM, less than about 60 μM, less than about 100 μM, or less than about 120 μM. Simvastatin lactone may be used to increase enzymatic activity of p38γ MAP kinase by at least about 20%, at least about 50%, at least about 100%, at least about 120%, at least about 127%, or at least about 140%.

In some embodiments, p38γ MAP kinase is activated by cerevastatin lactone. For example, p38γ MAP kinase can be activated by cerivastatin lactone administered to a concentration of less than about 3 μM, less than about 5 μM, less than about 10 μM, less than about 50 μM, less than about 60 μM, less than about 100 μM, or less than about 120 μM. Cerivastatin lactone may be used to increase enzymatic activity of p38γ MAP kinase by at least about 20%, at least about 50%, at least about 100%, at least about 115%, at least about 127%, or at least about 140%.

In some embodiments, p38γ MAP kinase is activated by rosuvastatin lactone. For example, p38γ MAP kinase can be activated by rosuvastatin lactone administered to a concentration of less than about 5 μM, less than about 10 μM, less than about 30 μM, less than about 50 μM, less than about 60 μM, less than about 100 μM, or less than about 120 μM. Rosuvastatin lactone may be used to increase enzymatic activity of p38γ MAP kinase by at least about 10%, at least about 15%, at least about 20%, at least about 25%, at least about 26%, or at least about 30%.

In some embodiments, p38γ MAP kinase is activated by atorvastatin lactone. For example, p38γ MAP kinase can be activated by atorvastatin lactone administered to a concentration of less than about 1 μM, less than about 3 μM, less than about 5 μM, less than about 8 μM, less than about 10 μM, or less than about 12 μM. Atorvastatin lactone may be used to increase enzymatic activity of p38γ MAP kinase by at least about 20%, at least about 40%, at least about 50%, at least about 60%, at least about 62%, or at least about 70%. In still some embodiments, p38γ MAP kinase is inhibited by atorvastatin lactone. For example, p38γ MAP kinase can be inhibited by atorvastatin lactone administered to a concentration of at least about 45 μM, at least about 50 μM, or at least about 55 μM.

In some embodiments, p38γ MAP kinase is activated by pitavastatin lactone. For example, p38γ MAP kinase can be activated by pitavastatin lactone administered to a concentration of less than about 3 μM, less than about 10 μM, less than about 30 μM, less than about 50 μM, less than about 60 μM, less than about 100 μM, or less than about 120 μM. Pitavastatin lactone may be used to increase enzymatic activity of p38γ MAP kinase by at least about 20%, at least about 50%, at least about 100%, at least about 150%, at least about 200%, at least about 240%, at least about 257%, or at least about 270%. In some embodiments, p38γ MAP kinase is not activated by fluvastatin lactone.

In some embodiments, p38δ MAP kinase is activated by simvastatin lactone. For example, p38δ MAP kinase can be activated by simvastatin lactone administered to a concentration of less than about 2 μM, less than about 4 μM, less than about 10 μM, less than about 30 μM, less than about 50 μM, less than about 60 μM, less than about 100 μM, or less than about 120 μM. Simvastatin lactone may be used to increase enzymatic activity of p38δ MAP kinase by at least about 20%, at least about 50%, at least about 100%, at least about 200%, at least about 300%, at least about 400%, at least about 500%, at least about 611%, or at least about 620%.

In some embodiments, p38δ MAP kinase is activated by cerivastatin lactone. For example, p38δ MAP kinase can be activated by cerivastatin lactone administered to a concentration of less than about 5 μM, less than about 8 μM, less than about 10 μM, less than about 30 μM, less than about 50 μM, less than about 60 μM, less than about 100 μM, or less than about 120 μM. Cerivastatin lactone may be used to increase enzymatic activity of p38δ MAP kinase by at least about 50%, at least about 60%, at least about 80%, at least about 100%, at least about 120%, at least about 150%, at least about 158%, at least about 160%, or at least about 170%.

In some embodiments, p38δ MAP kinase is activated by rosuvastatin lactone. For example, p38δ MAP kinase can be activated by rosuvastatin lactone administered to a concentration of less than about 10 μM, less than about 20 μM, less than about 30 μM, less than about 50 μM, less than about 60 μM, less than about 80 μM, less than about 100 μM, or less than about 120 μM. Rosuvastatin lactone may be used to increase enzymatic activity of p38δ MAP kinase by at least about 20%, at least about 30%, at least about 50%, at least about 60%, at least about 80%, at least about 100%, at least about 120%, at least about 138%, or at least about 145%.

In some embodiments, p38δ MAP kinase is activated by atorvastatin lactone. For example, p38δ MAP kinase can be activated by atorvastatin lactone administered to a concentration of less than about 20 μM, less than about 30 μM, less than about 50 μM, less than about 60 μM, less than about 100 μM, or less than about 120 μM. Atorvastatin lactone may be used to increase enzymatic activity of p38δ MAP kinase by at least about 20%, at least about 30%, at least about 50%, at least about 80%, at least about 100%, at least about 112%, or at least about 120%. In still some embodiments, p38δ MAP kinase is inhibited by atorvastatin lactone. For example, p38δ MAP kinase can be inhibited by atorvastatin lactone administered to a concentration of at least about 80 μM, at least about 83 μM, or at least about 85 μM.

In some embodiments, p38δ MAP kinase is activated by fluvastatin lactone. For example, p38δ MAP kinase can be activated by fluvastatin lactone administered to a concentration of less than about 1 μM, less than about 3 μM, less than about 10 μM, less than about 15 μM, less than about 30 μM, or less than about 35 μM. Fluvastatin lactone may be used to increase enzymatic activity of p38δ MAP kinase by at least about 20%, at least about 30%, at least about 50%, at least about 80%, at least about 100%, at least about 112%, or at least about 120%. In still some embodiments, p38δ MAP kinase is inhibited by fluvastatin lactone. For example, p38δ MAP kinase can be inhibited by fluvastatin lactone administered to a concentration of at least about 80 μM, at least about 90 μM, or at least about 100 μM.

In some embodiments, a compound described herein may be used to activate at least two MAP kinases. For example, the compound may be used to activate at least two MAP kinases selected from a p38α MAP kinase, a p38β MAP kinase, a p38γ MAP kinase, a p38δ MAP kinase, and a p42 MAP kinase. In some embodiments, the compound may be a known statin lactone, e.g., at least one lactone selected from simvastatin lactone, fluvastatin lactone, rosuvastatin lactone, and atorvastatin lactone. In some embodiments, a compound described herein may be used to activate a JNK MAP kinase, e.g., facilitating a Fas apoptotic pathway.

As Table I illustrates, simvastatin lactone and cerivastatin lactone can activate all four p38 MAP kinase isoforms, even at low micromolar concentrations and, in concentration dependent fashion, can achieve levels of activation (absolute efficacy) of about 611% above baseline. In some embodiments, e.g., at the concentrations of simvastatin lactone and cerivastatin lactone studied, activation can be achieved with no or substantially no accompanying inhibition of p38 MAP kinase isoforms α, β, γ, or δ.

In some embodiments, a mixed profile can be obtained, e.g., using fluvastatin lactone. As Table I illustrates, fluvastatin lactone can measurably activate three p38 MAP kinase isoforms (α, β, δ) at low to mid micromolar concentrations and can inhibit these same enzymes at higher concentrations. In particular, fluvastatin lactone activates p38δ MAP kinase with high potency (AC50 of about 3 μM) and high efficacy (e.g., about 440% activation at about 30 μM).

In some embodiments, a selective profile can be obtained. For example, as Table I illustrates, rosuvastatin lactone may not significantly inhibit or may not substantially inhibit p38 MAP kinases at or below about 100 μM, but may activate p38δ MAP kinase with AC50 of about 50 μM and an efficacy of about 138% at about 100 μM. At such concentration (at about 100 μM), rosuvastatin lactone may activate other p38 MAP kinase to a lesser degree, preferably to a much lesser degree in some selective embodiments. For example, rosuvastatin lactone only marginally activated p38γ MAP kinase and did not significantly or did not substantially activate either p38α MAP kinase, nor p38β MAP kinase.

In some embodiments, a more inhibitory profile can be obtained, e.g., using atorvastatin lactone. As Table I illustrates, atorvastatin lactone can display a more inhibitory profile towards the p38 MAP kinase isoforms α, β, or δ. For example, in some embodiments, IC50 values for atorvastatin lactone are below about 100 μM for p38 MAP kinase isoforms α, β, γ and δ. In some such embodiments, atorvastatin lactone may not or may not substantially activate p38α MAP kinase, e.g., at the concentrations studied, and may activate p38β MAP kinase only to a small degree. In still some embodiments, atorvastatin lactone may both inhibit and activate different MAP kinases, e.g., at different concentrations. For example, atorvastatin lacton showed both inhibitory and activation activities towards p38γ MAP kinase and p38δ MAP kinase, with AC50 values of about 10 μM to about 30 μM in some embodiments and with efficacies of about 112% in some embodiments.

III. Methods of Treatment

Another aspect of the present invention relates to methods of using pharmaceutical compositions and kits comprising compounds described herein to treat kinase-related and/or HMG-CoA reductase-related conditions, as well as novel uses of known compounds for the treatment of kinase-related conditions. In some embodiments, compositions and kits comprising a compound(s) described herein are used to modulate one or more types of kinases and/or HMG CoA reductase to provide different modulatory profiles towards different types of kinases, including various MAP kinase isoforms, as described above. Such different modulatory profiles find use in different medical applications, e.g., in the treatment of kinase-related and/or HMG-CoA reductase-related conditions, as described in detail below.

The present invention provides methods, pharmaceutical compositions, and kits for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying disorder being treated. For example, in an immuno-compromised patient, therapeutic benefit includes eradication or amelioration of immunocompromised status. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, a MAP kinase activator of the present invention provides therapeutic benefit not only when the status of being immunocomprised is eradicated, but also when an improvement is observed in the patient with respect to other effects, disorders, or discomforts that accompany immunocompromised status, like an increased immune response at least in some respects. Similarly, modulators of the present invention can provide therapeutic benefit in ameliorating other symptoms associated with kinase-related conditions, e.g., a parasitic infection, ischemic condition, diabetic condition and the like.

For prophylactic benefit, a pharmaceutical composition of the invention may be administered to a patient at risk of developing a kinase-related condition and/or an HMG-CoA reductase-related condition, or to a patient reporting one or more of the physiological symptoms of such conditions, even though a diagnosis of the condition may not have been made. Administration may prevent the condition from developing, or it may reduce, lessen, shorten and/or otherwise ameliorate the condition that develops.

A. Treatment of Kinase-Related Conditions

The term “kinase-related condition” as used herein refers to a condition in which directly or indirectly modulating the activity of one or more kinases is desired. The term “MAP kinase-related condition” as used herein refers to a condition in which directly or indirectly modulating the activity of a protein kinase involved in signaling cascades of an allergic, inflammatory and/or an autoimmune response is desirable, and/or directly or indirectly modulating the production and/or effects of one or more products of the protein kinase is desirable. In preferred embodiments, modulation involves direct activation of a protein kinase. For example, a MAP kinase-related condition may involve under-production of one or more pro-inflammatory cytokines, such as tumor necrosis factor-α (TNF-α), interleukin-1β (IL-1β), or other chemical messengers of signal transduction pathways associated with inflammation (including responses to and expression of TNF-α and IL-1β), apoptosis, growth and differentiation.

Examples of MAP kinase-related conditions include (but are not limited to) immuno-compromised conditions, hyperproliferative disorders, including cancer, infections and other parasitic conditions, ischemic conditions, and diabetic conditions. MAP-kinase related conditions can also include chronic obstructive pulmonary disease, as well as cardiovascular-related conditions such as atherosclerosis, myocardial infarction, congestive heart failure, thrombosis, myocardial infarction, ischemic-reperfusion injury and other vascular inflammatory conditions, including peripheral vascular diseases. Other conditions treatable with compositions, kits, and methods of the present invention include those currently treated with immunostimulants and/or activators of the mitogen-activated protein kinase (MAP kinase) family, preferably including conditions currently treated with activators of p38 MAP kinases and/or the stress-activated protein kinases/Jun N-terminal kinases (SAPKs/JNKs). Most preferably, conditions treatable with the practice of this invention include those relating to p38α MAP kinase, e.g., conditions currently treated or treatable by activation of p38α MAP kinase activity.

Some embodiments provide a method of treating a condition wherein an activation of a MAP kinase is desired comprising administering to a subject in need thereof a compound of formula I and/or II that modulates one or more types of MAP kinase. The compositions can exert modulatory effects in vitro and/or in vivo and can form the basis for pharmaceutical compositions useful in the treatment of MAP kinase-related conditions, e.g., immunocompromised conditions, in humans and other mammals. In certain embodiments, for example, these compositions improve production of, and signaling pathways involving, TNF-α and IL-1β, e.g., to help fight infection disease.

As noted above, a subset of the compounds of formulas I and II are novel analogs of known inhibitors of HMG-CoA reductase, wherein X comprises a lipophilic moiety of an HMG-CoA reductase inhibitor, e.g., a natural or synthetic statin, or an analog thereof, e.g., an analog comprising at least one moiety selected from a lactone moiety, a lactam moiety, a thiolactone moiety, a cyclic sulfonic ester moiety, a cyclic sulfuric ester moiety, a cyclic sulfonic amide moiety, a cyclic sulfuric amide moiety, a tetrahydropyran moiety, and a tetrahydrothiopyran moiety. Some of these novel analogs display a profile of activating and/or inhibiting activity in the lactone and/or acid forms, and are useful in the practice of this invention, e.g. in a method of treating a MAP kinase-related condition by administering to a subject an effective amount of at least one of such compounds. In some embodiments, the acid forms of such compounds also display MAP kinase inhibitory and/or MAP kinase activating activity. In some preferred embodiments, MAP kinase modulation, e.g., MAP kinase activation is direct. For example, in some embodiments, MAP kinase modulation is not reversed by addition of farnesyl pyrophosphate, geranyl geranyl pyrophosphate, mevalonate or any downstream product of mevalonate. In some embodiments, the lactone form does not inhibit or does not substantially inhibit HMG-CoA reductase. In some preferred embodiments, the lactone is not hydrolyzed, or not substantially hydrolyzed, to an acid form. In some such embodiments, the lactone does not inhibit or does not substantially inhibit HMG-CoA reductase. In some of these preferred embodiments, a lactone form may be formulated into solutions, suspensions, ointments and/or suppositories for topical application and/or rectal administration. In some embodiments, the acid forms of such compounds also display MAP kinase modulating activity.

Another subset of the compounds of formula I are known inhibitors of HMG-CoA reductase, including mevasatin, lovastatin, simvastatin, pravastatin, fluvastatin, atorvastatin, cerivastatin, rosuvastatin, pitavastatin, glenvastatin, bervastatin, dalvastatin, eptastatin, dihydroeptastatin, itavastatin, L-154819, advicor, L-654969, and other statin drugs used to treat disorders such as hypercholesterolemia and other lipid disorders. For example, the statin lactones illustrated in FIG. 3 are preferred in some embodiments for treating such MAP kinase-related conditions.

In the case of these compounds, the present invention relates to methods of using their corresponding lactones of formula I in novel treatments where MAP kinase modulation is desired, preferably where activating one or more p38 MAP kinases is desired, e.g., by administering an effective amount to a subject in need thereof. For example, in some embodiments where activation of p38α MAP kinase is desired, use of effective amounts of fluvastain lactone is preferred, and use of effective amounts of simvastatin lactone is more preferred. In some embodiments where activation of p38β MAP kinase is desired, use of effective amounts of atorvastatin lactone is preferred, use of effective amounts of fluvastatin lactone more preferred, and use of effective amounts of simvastatin lactone most preferred. In some embodiments where activation of p38γ MAP kinase is desired, use of effective amounts of rosuvastatin lactone is preferred, use of effective amounts of atorvastin lactone more preferred, use of effective amounts of simavastatin lactone even more preferred, and use of effective amounts of pitavastatin lactone most preferred. In some embodiments where activation of p38δ MAP kinase is desired, use of effective amounts of atorvastatin lactone are preferred, use of effective amounts of rosuvastatin lactone more preferred, use of effective amounts of fluvastatin lactone even more preferred, and use of effective amounts of simvastatin lactone most preferred.

In some preferred embodiments, MAP kinase modulation, e.g., MAP kinase activation is direct, e.g., the activation does not occur via a growth factor, a cytokine receptor and/or environmental stress. For example, in some embodiments, MAP kinase modulation is not reversed by addition of farnesyl pyrophosphate, geranyl geranyl pyrophosphate, mevalonate or any downstream product of mevalonate. In some embodiments, the lactone form does not inhibit or does not substantially inhibit HMG-CoA reductase. In some preferred embodiments, the lactone is not hydrolyzed, or not substantially hydrolyzed, to an acid form. In some such embodiments, the lactone does not inhibit or does not substantially inhibit HMG-CoA reductase. In some embodiments, the activation occurs in a cell other than a brain cell. For example, in some embodiments the composition used does not increase the levels and/or activities of protein kinases in brain cells, e.g., in brain cells in a culture. See Lynch et al. (U.S. 2002/0048746). In some embodiments, the activation does not involve a protein kinase C pathway. See Gasper et al. (U.S. 2001/0034364). In some of these preferred embodiments, a lactone form may be formulated into solutions, suspensions, ointments and/or suppositories for topical application and/or rectal administration. In some embodiments, the acid forms of such compounds also display MAP kinase modulating activity.

For instance, compounds described herein that activate p38α MAP kinase find use as immunostimulants, e.g., in treating an immunocompromised condition by administering an effective amount to a subject in need thereof. The p38α isoform is known to be closely associated with immune and inflammatory signally pathways leading to expression and action of pro-inflammatory cytokines (e.g., IL-1β and TNF-α). See, e.g., Ono et al., Cell. Signal 12, 1-13 b(2000); Kiener et al., Intl. Immunopharmacol. 1, 105-118 (2001). The term “immuno-compromised condition” refers to the condition of an immuno-compromised subject, e.g., a subject having an immune system which is compromised, at least in part. The immuno-compromised status can be due to a genetic disorder, disease or drugs that inhibit the immune response. The compromise can be temporary or permanent. An immuno-compromised subject can include individuals who are afflicted with cystic fibrosis, HIV, Hepatitis B and C, other infectious diseases, or who are taking corticosteroids or immunosuppressive agents. In preferred embodiments, p38α MAP kinase activation is direct. Some preferred embodiments use simvastatin lactone and/or fluvastatin lactone administered in an effective amount. In some embodiments, p38α MAP kinase is activated by lovastatin lactone and/or mevastatin lactone administered in an effective amount.

In some embodiments, compounds described herein that activate a JNK MAP kinase find use in treatment of hyperproliferative conditions, including (but not limited to) cancer. In some embodiments, compounds described herein that activate at least two MAP kinases (e.g., more than one p38 and/or other MAP kinases) find use in treatment of hyperproliferative conditions, including (but not limited to) cancer. In some embodiments, the at least two MAP kinases are selected from a p38α MAP kinase, a p38β MAP kinase, a p38γ MAP kinase, a p38γ MAP kinase, and a p42 MAP kinase. In some embodiments, treatment does not involve use of a polyene macrolide antibiotic. See Solomon (WO 03/086418).

As used herein, the term “cancer” can refer to any type of cancer such as leukemia (e.g., acute lymphocytic leukemia, acute myeloid leukemia, chronic myeloid leukemia and chronic lymphocytic leukemia), gastrointestinal carcinoid tumors, malignant mesothelioma, lung cancer, colon cancer, CNS cancer, melanoma, ovarian cancer, renal cancer, prostate cancer, multiple myeloma, Hodgkin lymphoma and non-Hodgkin lymphoma, melanomas of the skin, as well as cancer of the breast, prostate, lung and/or bronchus, colon and/or rectum, urinary bladder, kidney and/or renal pelvis, pancreas, oral cavity and/or pharynx (head & neck), ovary, thyroid, stomach, brain, esophagus, liver and/or intrahepatic bile duct, cervix, larynx, soft tissue including heart, testis, small intestine, anus, anal canal and/or anorectum, vulva, gallbladder, pleura, bones and/or joints, hypopharynx, eye and/or orbit, nose, nasal cavity and/or middle ear, nasopharynx, ureter, peritoneum, omentum and/or mesentery, and the like. Broad-based activation of MAP kinase pathways is known to promote programmed cell death (apoptosis) of the hyperproliferative cells. See, e.g., Curtin et al., Br. J. Cancer 87, 1188-1194 (2002). Such activators can be administered in combination with chemotherapeutic agents and/or with other molecularly targeted agent for treating hyperproliferative conditions. In preferred embodiments, MAP kinase activation is direct. Some preferred embodiments use rosuvastatin, more preferably fluvastatin and/or atrovastatin, and/or most preferably simvastatin in effective amounts. Activators of JNK MAP kinase are preferred with respect to treatment of prostate cancer.

In some embodiments, p38 MAP kinase activators described herein find use in treating parasitic conditions. The term “parasitic conditions” can refer to a condition resulting from infection with a parasitic organism, including, for example, toxoplasmosis (adult and infant forms), malaria, African sleeping sickness, Chagas disease, typhoid fever, typhus, worm conditions, fluke infections, insect parasite conditions, helminth infections, protozoan infections, schistosomiasis, filariasis, trypanosomiasis, leishmaniasis and the like. Preferred embodiments are directed at leishmanicidal agents. See, e.g., Awasthi et al., J. Exp. Med. 197, 1037-1043 (2003); Bagi (WO 01/37876). Parasitic conditions need not include an infection due, or at least partly due, to a bacterium, e.g., an intra cellular vacuolar bacterium. See Catron et al. (U.S. 2003/0087430).

In some embodiments, p38 MAP kinase activators described herein find use in treating ischemic conditions, such as oxidative and other stresses related to ischmia. In preferred embodiments, for example, p38 MAP kinase activators described herein can be used to protect the liver and/or other organs from ischemia-reperfusion injury and related stresses. See, e.g., Schauer et al., Hepatology 37, 286-295 (2003). Ischemic conditions may also include neurodegenerative diseases, e.g. CNS and peripheral neuropathies, ALS, Parkinson's, Alzheimer's and pain sensation. Ischemic conditions may also include cerbrovascular ischemia, ischemic cardiomyopathy, limb ischemia, myocardial ischemia, pulmonary ischemia, renal ischemia, and ischemia of tissues, such as muscle, kidney and lung. In the case of compositions comprising known statin lactones, treatment of an ischemic condition preferably involves administering a known statin lactone to provide a prophylactic treatment of the ischemic condition. Prophylactic treatment of the ischemic condition refers to treatment administered prior to an ischemic event, e.g., administering a known lactone prior to or during a stroke, as opposed to after a stroke has occurred; and/or administering a known lactone or other compound described herein prior to aortic or transplantation surgery as opposed to after such procedures. See Chopp (WO 03/086379); Joyce et al., J. of Surgical Research, 101, 79-84 (2001). In some embodiments, treatment of an ischemic condition need not include treatment involving activation of Akt, e.g., Ak/PKBt in vascular endothelial cells. See Walsh (U.S. 2004/0122077); Walsh (WO/0193806).

In some embodiments, p38 MAP kinase activators described herein find use in treating diabetic conditions. By “diabetic condition” is meant a condition relating to improper glucose uptake, including, e.g., type 1 diabetes, type 2 diabetes and/or gestational diabetes. For example, improper glucose uptake due to lack of activation of a glucose transporter can result in a diabetic condition, e.g., lack of activation of GLUT1, a housekeeping isoform of mammalian glucose transporters, can result in a GLUT1-related diabetic condition. Without being limited to a particular hypothesis, activation of p38 MAP kinase under the practice of the present invention can activate GLUT1 to treat such a condition, e.g., by stimulating glucose uptake. Diabetic conditions may also include diabetic nephropathy malignant nephrosclerosis. See, e.g., Barros et al., J. Physiol. 504, 517 525 (1997). In preferred embodiments, MAP kinase activation is direct.

Further, certain analogs of known lipophilic HMG-CoA reductase inhibitors having structures modified to favor a closed ring structure or cyclic form, including compounds of formulas III and IV described above, can also display MAP kinase modulating activity. Such structures can be useful in the practice of this invention, e.g., in a method of treating a MAP kinase-related condition by administering to a subject an effective amount of at least one of such compounds. In some embodiments, a compound of formula III or IV does not inhibit or does not substantially inhibit HMG-CoA reductase. More preferred embodiments include des-oxo and δ-lactam derivatives from a statin, e.g., metvastatin derivatives, lovostatin derivatives, simvastatin derivatives, atorvastatin derivatives, fluvastatin derivatives, rosuvastatin derivatives, cerivastatin derivatives, pitavastatin derivatives and/or glenvastatin derivatives, as described above.

The present invention also includes kits that can be used to treat a MAP kinase-related condition. These kits comprise a compound or compounds described herein and preferably instructions teaching the use of the kit according to the various methods and approaches described herein. Such kits also include information, such as scientific literature references, package insert materials, in vitro results, clinical trial results, and/or summaries of these and the like, which indicate or establish the activities, modulatory profiles, and/or advantages of the composition. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

A. Treatment of HMG-CoA Reductase-Related Conditions

The term “HMG-CoA reductase-related condition” as used herein refers to a condition in which directly or indirectly modulating, e.g., reducing, the activity of HMG-CoA reductase is desirable and/or directly or indirectly modulating, e.g., reducing, the production and/or effects of one or more products of HMG-CoA reductase is desirable. For example, an HMG-CoA reductase-related condition may involve elevated levels of cholesterol, in particular, non-HDL cholesterol in plasma (e.g., elevated levels of LDL cholesterol). Typically, a patient is considered to have high or elevated cholesterol levels based on a number of criteria (See, e.g., Pearlman B L, Postgrad Med 112(2):13-26 (2002), incorporated herein by reference). Guidelines include serum lipid profiles, such as LDL compared with HDL levels.

Examples of HMG-CoA reductase-related conditions include hypercholesterolemia, lipid disorders such as hyperlipidemia, and atherogenesis and its sequelae of cardiovascular diseases, including atherosclerosis, other vascular inflammatory conditions, myocardial infarction, ischemic stroke, occlusive stroke, and peripheral vascular diseases, as well as other conditions in which decreasing cholesterol and/or other products of the cholesterol biosynthetic pathways can produce a benefit. Other HMG-CoA reductase-related conditions treatable with compositions, kits, and methods of the present invention include those currently treated with statins.

Reducing the activity of HMG-CoA reductase, is also referred to as “inhibiting” the enzyme. The term “inhibits” and its grammatical conjugations, such as “inhibitory,” do not require complete inhibition, but refer to a reduction in HMG-CoA reductase activity. Such reduction is preferably by at least about 50%, at least about 75%, at least about 90%, and more preferably by at least about 95% of the activity of the enzyme in the absence of the inhibitory effect, e.g., in the absence of an inhibitor. Conversely, the phrase “does not inhibit” and its grammatical conjugations refer to situations where there is less than about 20%, less than about 10%, and preferably less than about 5% of reduction in enzyme activity in the presence of the compound. Further the phrase “does not substantially inhibit” and its grammatical conjugations refer to situations where there is less than about 30%, less than about 20%, and preferably less than about 10% of reduction in enzyme activity in the presence of the compound.

The ability to reduce enzyme activity is a measure of the potency or the activity of the compound towards or against the enzyme. Potency is preferably measured by cell free, whole cell and/or in vivo assays in terms of IC50 or ED50 values. An IC50 value represents the concentration of a compound required to inhibit the enzyme activity by half (50%) under a given set of conditions. A Ki value represents the equilibrium affinity constant for the binding of an inhibiting compound to the enzyme. An ED50 value represents the dose of a compound required to effect a half-maximal response in a biological assay. Further details of these measures will be appreciated by those of ordinary skill in the art, and can be found in standard texts on biochemistry, enzymology, and the like.

In some embodiments, compounds in one or more forms represented by formulas I, II, III, and IV inhibit HMG-CoA reductase. In many embodiments, compounds of formula II inhibit HMG-CoA reductase. Such compounds find use in the practice of this invention e.g., in a method of treating an HMG-CoA reductase-related condition by administering to a subject an effective amount of at least one of such compounds. These compositions can lower cholesterol levels in vitro and in vivo and form the basis for pharmaceutical compositions useful in the treatment of HMG-CoA reductase-related conditions, e.g., hypercholesterolemia and atherosclerosis, in humans and other mammals.

As noted above; a subset of the compounds of formulas I and II are novel analogs of known inhibitors of HMG-CoA reductase, wherein X comprises a lipophilic moiety of an HMG-CoA reductase inhibitor, e.g., a statin, or an analog thereof. Some of these analogs retain HMG-CoA reductase inhibitory activity in the lactone and/or acid form, in particular, in the acid carboxylate form, and are useful in the practice of this invention, e.g., in a method of treating an HMG-CoA reductase-related condition by administering to a subject an effective amount of at least one of such compounds. For example, acid carboxylate forms of certain lactone derivatives of statins illustrated in FIG. 3 are preferred in some embodiments.

The present invention also includes kits that can be used to treat an HMG-CoA reductase-related condition. These kits comprise a compound or compounds described herein, and preferably instructions teaching the use of the kit according to the various methods and approaches described herein. Such kits also include information, such as scientific literature references, package insert materials, in vitro results, clinical trial results, and/or summaries of these and the like, which indicate or establish the activities, modulatory profiles and/or advantages of the composition. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

B. Treatment of Both MAP Kinase- and HMG-CoA Reductase-Related Conditions

One of the purposes of this invention is to describe compounds which modulate both kinases (e.g., MAP kinases) and HMG-CoA reductase. For example, in some embodiments, a compound described herein may modulate the activity of different types of kinases in different ways, e.g., activating one or more types of MAP kinase, inhibiting one more types of MAP kinase, and inhibiting HMG-CoA. Such compounds can exert concomitant immune-boosting, anti-inflammatory and cholesterol-lowering effects in vitro and/or in vivo. In certain embodiments, for example, these compositions increase cholesterol uptake, increase HDL levels, reduce production of, and signaling pathways involving, TNF-α and IL-1β, as well as inhibiting production of cholesterol and/or other downstream products of mevalonate, including mevalonate pyrophosphate, isopentyl pyrophosphate, geranyl pyrophosphate, farnesyl pyrophosphate, dolichols, farnesylated proteins, trans-trans geranylgeranyl pyrophosphate, ubiquinone, geranyl-geranylated proteins, squalene, and the like. Further, in some embodiments, these compositions can exert superior anti-atherogenesis and/or anti-inflammatory effects in vivo, for example improving serum lipoprotein profiles, such as LDL compared with HDL levels.

Such compounds can form the basis for pharmaceutical compositions, kits, and methods for treating both MAP kinase-related conditions and HMG-CoA reductase-related conditions in humans and other animals. Moreover, such compositions can provide superior benefits in treating HMG-CoA reductase-related conditions, such as cardiovascular disease, compared with treatments that inhibit HMG-CoA reductase but do not activate or do not substantially activate a MAP kinase, or that do not inhibit or do not substantially inhibit a MAP kinase. Also, compositions of the present invention can provide superior benefits in treating MAP kinase-related conditions, such as inflammatory conditions, compared with treatments that modulate MAP kinases but do not inhibit or do not substantially inhibit HMG-CoA reductase.

In some embodiments, the closed-ring form (formula I) of a compound of this invention can modulate a MAP kinase, and the corresponding open form (formula II), in particular the deprotonated carboxylate form, can inhibit HMG-CoA reductase. Accordingly, this treatment approach can provide a benefit in both a HMG-CoA reductase-related condition and a MAP kinase-related condition, for instance, in a method comprising administering to a subject an effective amount of at least one of such compounds, e.g., improving serum lipid profiles and reducing cholesterol production in the treatment of a HMG-CoA reductase-related condition, such as cardiovascular disease. Improved serum lipid profiles by modulation of more than one MAP kinase, e.g., inhibiting p38α MAP kinase and activating a non-p38α MAP kinase, may be in addition to other immunomodulatory effects of some HMG-CoA reductase inhibitors that, for example, produce immunomodulatory responses though the action of metabolites such as farnesyl pyrophosphate and/or geranylgeranyl pyrophosphate. Moreover, in some embodiments, the role of a compound of the present invention in activating one or more MAP kinases and/or inhibiting one or more other MAP kinases is distinct from the anti-inflammatory effects of some statins through reducing the synthesis of metabolite products such as geranylgeranyl pyrophosphate and/or farnesyl pyrophosphate. For example, the modulatory activity of some compounds of this invention on a MAP kinase and on MAP-kinase related conditions need not be reversed by exogenous addition of mevalonate (e.g., sodium-mevalonate), geranylgeranyl pyrophosphate and/or farnesyl pyrophosphate, and/or other downstream product of mevalonate.

Furthermore, the interplay between inflammatory and HMG-CoA reductase-related disorders means that compositions regulating both a MAP kinase and HMG-CoA reductase pathways can be particularly beneficial. Inhibition of HMG-CoA reductase can lead to improved serum lipoprotein profiles, such as decreased LDL and increased HDL levels, which in turn can lead to a reduction in the rate of atherogenesis. Similarly, activation of MAP kinases other than p38α MAP kinase can serve to increase expression of LDL receptors on the surface of liver cells, affording a further decrease in LDL levels (see, e.g., Dhawan et al., J. Lipid Res. 40, 1911-1919 (1999)) as well as increased production of protective HDL (see, e.g., Nofer et al., J. Biol. Chem. 278, 53055-53062 (2003)). Synergistically, initiation of atherogenic plaque deposition (e.g., via foam cells) is reduced by the anti-inflammatory effects, including those which derive from inhibition of p38α MAP kinase. Inhibition of p38α MAP kinase can also antagonize inflammatory processes which contribute to the progression and rupture of atherogenic plaques and which, in turn, can lead to arterial thrombosis, blockade, etc. See, e.g., Palinski et al., J. Am. Soc. Nephrol. 13, 1673-1681 (2002). Consequently, pharmaceutical compositions including a compound of formula I/II and having modulatory activity towards both a MAP kinase and HMG-CoA reductase can be syngerigsic, superior, and preferably differentially superior, to drugs targeting only HMG-CoA reductase. In some preferred embodiments, such compositions can provide a differentially superior benefit in treating cardiovascular disease related to atherogenesis, including formation and disruption of atherosclerotic plaques.

In some embodiments, a compound of formula I modulates or is more potent towards a MAP kinase while the corresponding compound of formula II, with equivalent stereochemistry, inhibits or is more potent against HMG-CoA reductase. In some preferred embodiments, the activities or potencies of a compound of formula I and the corresponding compound of formula II are similar towards one or more MAP kinases and HMG-CoA reductase. In other preferred embodiments, the potency of a compound of formula I and/or II towards one or more MAP kinases is greater than its potency against HMG-CoA reductase. In yet other preferred embodiments, the potency of a compound of formula I and/or II against HMG-CoA reductase is greater than its potency towards one or more MAP kinases. In some embodiments, compounds of formulas I and II having absolute configuration illustrated in the structures of FIG. 3 are preferred.

In some embodiments, a compound of formula I modulates both one or more MAP kinases and HMG-CoA reductase. In some embodiments, a compound of formula II modulates both one or more MAP kinases and HMG-CoA reductase. In other embodiments, a compound of formula III modulates both one or more MAP kinases and HMG-CoA reductase; in still other embodiments, a compound of formula IV modulates both one or more MAP kinases and HMG-CoA reductase. In nearly all preferred embodiments for treating a cardiovascular condition, compounds of formula II inhibit HMG-CoA reductase.

As noted above, a subset of the compounds of formulas I and II are novel analogs of known inhibitors of HMG-CoA reductase, wherein X comprises a lipophilic moiety of an HMG-CoA reductase inhibitor, e.g., a statin, a natural or synthetic statin, or an analog thereof, e.g., an analog comprising at least one moiety selected from a lactone moiety, a lactam moiety, a thiohactone moiety, a cyclic sulfonic ester moiety, a cyclic sulfuric ester moiety, a cyclic sulfonic amide moiety, a cyclic sulfuric amide moiety, a tetrahydropyran moiety, and a tetrahyddrothiopyran moiety. Some of these analogs retain HMG-CoA reductase inhibitory activity in the acid and/or lactone forms while also exhibiting MAP kinase modulatory activity/activities in the lactone and/or acid forms. In some preferred embodiments, a statin analog of the present invention inhibits HMG-CoA reductase in the acid form (Formula II, in particular, in the carboxylate form) and modulates one or more MAP kinases in the corresponding lactone form (Formula I). For example, in some embodiments, the lactone form of the compound activates a non-p38α MAP kinase and/or inhibits a p38α MAP kinase, whereas the acid and/or salt form of the compound inhibits HMG-CoA reductase, preferably in a liver-selective manner. For example, the non-p38α MAP kinase can be p42/44 MAP kinase and/or JNK. In preferred embodiments, such compounds find use in the practice of the invention, e.g., in a method comprising administering to a subject an effective amount of at least one of such compounds to treat a MAP kinase-related condition and/or treating an HMG-CoA reductase-related condition. Analogs of statins illustrated in FIG. 3 can provide examples of such preferred embodiments. In other embodiments, the lactone form modulates one or more MAP kinases and HMG-CoA reductase; in still other embodiments, the acid form modulates one or more MAP kinases and HMG-CoA reductase.

The present invention also includes kits that can be used to treat kinase- and HMG-CoA reductase-related conditions, in particular cardiovascular disease related to atherogenesis. These kits can comprise a compound or compounds described herein, including compounds of formula I and/or II which have modulatory activity towards one or more MAP kinases and towards HMG-CoA reductase, and preferably instructions teaching the use of the kit according to the various methods and approaches described herein. Such kits also include information, such as scientific literature references, package insert materials, in vitro results, clinical trial results, and/or summaries of these, and the like, which indicate or establish the multiple activities of the composition and indicate and/or establish how use of the composition provides modulatory profiles, advantages and/or differential superiority in treating an HMG-CoA reductase- and/or a MAP kinase-related condition, preferably in treating cardiovascular disease. Such information may be based on the results of various studies, for example, studies using experimental animals involving in vivo models and studies based on human clinical trials. Kits of the present invention may also include materials comparing the approaches of the present invention with other therapies, which do not display a combination of MAP kinase plus HMG-CoA reductase modulatory activities. Kits described herein can be provided, marketed and/or promoted to health providers, including physicians, nurses, pharmacists, formulary officials, and the like.

IV. Formulations, Routes of Administration, and Effective Doses

Yet another aspect of the present invention relates to pharmaceutical compositions comprising a modulator of a kinase and/or HMG-CoA reductase. Such pharmaceutical compositions can be used to treat kinase-related and/or FMG-CoA reductase-related conditions, as described in detail above.

The compounds of formula I/II may be provided in either the closed or open form, and/or may be allowed to interconvert in vivo after administration. For example, either δ-lactone or hydroxy carboxylic acid forms, or pharmaceutically acceptable salts, esters or amides thereof, may be used in developing a formulation for use in the present invention. Further, in some embodiments, the compound may be used in combination with one or more other compounds or in one or more other forms. For example a formulation may comprise both the closed and open forms in particular proportions, depending on the relative potencies of the closed and open forms and the intended indication. For example, in compositions for treating MAP kinase- and/or HMG-CoA reductase-related conditions where a lactone form modulates one or more MAP kinases and an acid (carboxylate) form inhibits HMG-CoA reductase, and where potencies are similar, about a 1:1 ratio of lactone to acid forms may be used. The two forms may be formulated together, in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

Similarly, compounds of formula III and IV, or their pharmaceutically acceptable salts, esters, or amides thereof, may be used alone, together, or in combination with the corresponding or other compounds of formula I and II, described above. For example, a compound of formula IV (closed δ-lactam ring) may be co-administered with a compound of formula II (open acid form), where the compounds have equivalent stereochemistries. Such administration may be useful for treating both MAP kinase- and HMG-CoA reductase-related conditions, for example, where the lactam form modulates one or more MAP kinases and the acid (carboxylate) form inhibits HMG-CoA reductase. The two forms may be formulated together, in the same dosage unit e.g. in one cream, suppository, tablet, capsule, or packet of powder to be dissolved in a beverage; or each form may be formulated in separate units, e.g., two creams, suppositories, tablets, two capsules, a tablet and a liquid for dissolving the tablet, a packet of powder and a liquid for dissolving the powder, etc.

The term “pharmaceutically acceptable salt” means those salts which retain the biological effectiveness and properties of the compounds used in the present invention, and which are not biologically or otherwise undesirable. For example, a pharmaceutically acceptable salt does not interfere with the beneficial effect of the compound used in the invention in modulating MAP kinase and/or HMG-CoA reductase, e.g., in treating a MAP kinase and/or HMG-CoA reductase related condition.

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide refers to those which retain biological effectiveness and properties of the compounds used in the present invention, and which are not biologically or otherwise undesirable. For example, the ester or amide does not interfere with the beneficial effect of the compound of the invention in modulating one or more MAP kinases and/or inhibiting HMG-CoA reductase. Typical esters include ethyl, methyl, isobutyl, ethylene glycol, and the like. Typical amides include unsubstituted amides, alkyl amides, dialkyl amides and the like.

If necessary or desirable, the modulator may be administered in combination with other therapeutic agents. The choice of therapeutic agents that can be co-administered with the compositions dicussed herein can depend, at least in part, on the condition being treated. For example, in some embodiments for treating hyperporliferative conditions, a compound described herein can be administered in combination with chemotherapeutic agents and/or with other molecularly targeted agents. In some embodiments for treating immunocompromised conditions, a compound described herein can be administered in combination with other immunostimulants. Examples of immunostimulants include adjuvants, biodegradable microspheres, e.g., polylactic galactide, liposomes (into which the compound(s) can be incorporated), and the like. Also, in some embodiments, a compound described herein may be administered with at least one compound selected from farnesyl pyrophosphate, geranylgeranyl pyrophosphate, mevalonate and/or a downstream product of mevalonate. Such co-administration may be desirable to reduce the action of the compound as an HMG-CoA reductase inhibitor, e.g., making the compound more specific in terms of its MAP kinase modulatory effects. Agents of particular use in the formulations used in the present invention include, for example, any agent having a therapeutic effect for kinase-related and/or HMG-CoA reductase related conditions.

The modulators (or pharmaceutically acceptable salts, esters or amides thereof) may be administered per se or in the form of a pharmaceutical composition wherein the active compound(s) is in an admixture or mixture with one or more pharmaceutically acceptable carriers. A pharmaceutical composition as used herein may be any composition prepared for administration to a subject. Pharmaceutical compositions for use in accordance with the present invention may be formulated in conventional manner using one or more physiologically acceptable carriers comprising excipients, diluents and/or auxiliaries, e.g., which facilitate processing of the active compounds into preparations that can be administered. Proper formulation may depend at least in part upon the route of administration chosen. The modulators useful in the present invention, or pharmaceutically acceptable salts, esters, or amides thereof, can be delivered to the patient using a number of routes or modes of administration, including oral, buccal, topical, rectal, transdermal, transmucosal, subcutaneous, intravenous, and intramuscular applications, as well as by inhalation.

For oral administration, the compounds can be formulated readily by combining the active compound(s) with pharmaceutically acceptable carriers well known in the art. Such carriers enable the compounds used in the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the compounds used in the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use may contain compound(s) described herein with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the compounds into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, e.g., Bangham et al., J. Mol. Biol. 23: 238-252 (1965); Szoka et al., Proc. Natl. Acad. Sci. USA 75: 4194-4198 (1978). Ligands may also be attached to the liposomes to direct these compositions to particular sites of action. Compounds of this invention may also be integrated into foodstuffs, e.g., cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

For injection, the modulators of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P. These compounds may also be formulated for transmucosal administration, buccal administration, for administration by inhalation, for parental administration, for transdermal administration, and rectal administration.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In some preferred embodiments, the compounds used in the present invention are delivered in soluble rather than suspension form, which allows for more rapid and quantitative absorption to the sites of action. In general, formulations such as jellies, creams, lotions, suppositories and ointments can provide an area with more extended exposure to the compounds used in the present invention, while formulations in solution, e.g., sprays, provide more immediate, short-term exposure.

In some embodiments, the pharmacuetical compositions can include one or more penetration enhancers. For example, the formulations may comprise suitable solid or gel phase carriers or excipients that increase penetration or help delivery of compound(s) across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, α-bisabolol, d-limonene, urea and N,N-diethyl-m-toluamide, and the like Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In some embodiments, the pharmaceutical compositions will include one or more such penetration enhancers.

In some embodiments, the pharmaceutical compositions for local/topical application can include one or more antimicrobial preservatives such as quaternary ammonium compounds, organic mercurials, p-hydroxy benzoates, aromatic alcohols, chlorobutanol, and the like.

Direct topical application, e.g., of a viscous liquid, gel, jelly, cream, lotion, ointment, suppository, foam, or aerosol spray, may be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry, (ed.), (Marcel Dekker Incl, 1983).

Additional details of formulations for use in some embodiments of the instant invention arre provided in Example 4 below.

Dosages

Pharmaceutical compositions suitable for use in the present invention include compositions wherein the active ingredients are present in an effective amount, i.e., in an amount effective to achieve therapeutic and/or prophylactic benefit in at least one of a MAP kinase-related condition and an HMG-CoA reductase-related condition. The actual amount effective for a particular application will depend on the condition or conditions being treated, the condition of the subject, the formulation, and the route of administration, as well as other factors known to those of skill in the art. Determination of an effective amount of a MAP kinase and/or HMG-CoA reductase modulator is well within the capabilities of those skilled in the art, in light of the disclosure and experimental results herein, and will be determined using routine optimization techniques.

The effective amount for use in humans can be determined from animal models. For example, a dose for humans can be formulated to achieve circulating, liver, topical and/or gastrointestinal concentrations that have been found to be effective in animals.

The effective amount when referring to an modulator of the invention will generally mean the dose ranges, modes of administration, formulations, etc., that have been recommended or approved by any of the various regulatory or advisory organizations in the medical or pharmaceutical arts (e.g., FDA, AMA) or by the manufacturer or supplier. Effective amounts of HMG-CoA reductase inhibitors can be found, for example, in the Physicians Desk Reference. For example, daily doses for atorvastatin calcium range from about 2 mg to about 50 mg, from about 3 mg to about 30 mg, typically about 10 mg. A daily dose for cerivastatin sodium is about 200 μg, while daily doses for fluvastatin sodium, rosuvastatin sodium, pravastatin sodium and simvastatin are each about 20 mg. Some preferred compounds of this invention, e.g., analogs of HMG-CoA reductase inhibitors, may be useful in about the same dosages, or less than, or more than dosages typical of known HMG-CoA reductase inhibitors.

Generally, the recommended dosage for an HMG-CoA reductase inhibitor of the present invention is a dose of about 0.01 mg/kg to about 1,000 mg/kg, more preferably from about 0.1 mg/kg to about 20 mg/kg on a daily basis, provided orally. The inhibitor is typically administered in a dose of about 10 mg, which is in the range of doses that will be useful in the present invention. Using other routes of administration, it is believed that a dose of about 0.01 mg/kg/day to about 1,000 mg/kg/day of an HMG-CoA reductase inhibitor will be used; preferably a dose between about 0.1 mg/kg/day and about 1 mg/kg/day will be used.

Effective amounts of MAP kinase modulators, e.g., MAP kinase inhibitors or acitvators, can be found, for example, in published reports of the results of human clinical trials. Generally, the recommended dosage for a MAP kinase modulator of the present invention, e.g., a p38α MAP kinase inhibitor, is a dose of about 0.01 mg/kg to about 1,000 mg/kg, more preferably from about 0.1 mg/kg to about 20 mg/kg on a daily basis, provided orally. The inhibitor is typically administered in a dose of about 100 mg, which is in the range of doses that will be useful in the present invention. Using other routes of administration, it is believed that a dose of about 0.01 mg/kg/day to about 1,000 mg/kg/day of a MAP kinase modulator will be used; preferably a dose between about 0.1 mg/kg/day and about 20 mg/kg/day will be used.

Further, appropriate doses for a MAP kinase modulator can be determined based on in vitro experimental results provided herein. For example, the in vitro potency of a compound in activating one or more MAP kinase isoforms and/or inhibiting one or more MAP kinase isoforms provides information useful in the development of effective in vivo dosages to achieve similar biological effects. Table I above, for example, provides such data.

In some embodiments, administration of compounds for use in the present invention may be intermittent, for example administration about once every two days, about every three days, about every five days, about once a week, about once or twice a month, and the like. In some embodiments, the amount, forms, and/or amounts of the different forms may be varied at different times of administration. For example, at one point in time, the open or acid form of a compound of the present invention may be administered, while at another time the corresponding closed or lactone form may be used.

A person of skill in the art would be able to monitor in a patient the effect of administration of a particular compound. For example, cholesterol levels can be determined by measuring LDL, HDL, and/or total serum cholesterol levels. The release of pro-inflammatory cytokines can be determined by measuring TNF-α and/or IL-1β. Other techniques would be apparent to one of skill in the art.

V. Rational Design of Kinase and/or HMG-CoA Reductase Modulators

Still another aspect of the present invention relates to methods of obtaining and/or making a composition for modulating one or more kinases and/or HMG-CoA reductase by designing a compound of formula I/II. Some embodiments provide a method of making a composition for modulating a MAP kinase and/or inhibiting an HMG-CoA reductase by designing a compound of formula I/II; testing whether the compound activates a MAP kinase (e.g., by direct activation); inhibits the same or a different MAP kinase, and/or inhibits HMG-CoA reductase; and using the compound in making a composition for modulating one or more MAP kinase(s) and/or HMG-CoA reductase. More preferably, the invention relates to methods for designing and testing compounds of formula I that are capable of modulating one or more MAP kinase(s) to produce a profile of modulation with respect to different kinase isoforms

By “formula I/II” it is meant that either the closed (e.g., lactone) form or the open (e.g., hydroxy carboxylic acid) form, or both forms, may be responsible for modulation of a MAP kinase, HMG-CoA reductase or both.

For example, in some embodiments, known inhibitors of HMG-CoA reductase are systematically varied and tested for MAP kinase modulatory activity. In this approach, lipophilic moieties (X) of known inhibitors of HMG-CoA reductase are systematically varied, resulting in analogs of Formula I, e.g., to provide a range of activating and/or inhibitory profiles towards MAP kinases. FIG. 3, for example, illustrates known statins that can be used in designing some preferred embodiments. In some embodiments, the lipophilic moiety analog is selected on the basis of structural diversity or similarity to lipohilic moieties X of HMG-CoA reductase inhibitors and/or to a MAP kinase modulator, preferably to a lipophilic moiety of a MAP kinase activator. In some embodiments, the lipophilic moiety analog is selected on the basis of structural compatibility with binding to HMG-CoA reductase and/or to a MAP kinase, such as p38α MAP kinase, for example, using pharmacophore modeling to indicate binding compatibility.

The rational design methods of the present invention are aided by the current understanding of the crystal structures of HMG-CoA reductase and MAP kinases. The X-ray structure of p38α MAP kinase, for example, has been shown to comprise an N-terminal domain with an ATP binding pocket, and a C-terminal domain with a catalytic site, metal binding site, and phophorylation lip. The two domains are connected by a hinge region, to which the substrate binds. Further, a direct correlation has been shown between the “tightness” of binding of a candidate compound to the kinase macromolecule and the in vitro cellular activity of the compound. With respect to HMG-CoA reductase, most known statins, bind to the enzyme through the agency of at least two distinct substructures: a 3,5-dihydroxyheptanoate (or 3,5-dihydroxyhept-6-enoate) side chain and an attached lipophilic moiety. The dihydroxy carboxylate side chain mimics the 3-hydroxy-3-methyl glutaryl group of the natural substrate, while the lipophilic moiety interacts with a hydrophobic binding pocket on the enzyme which otherwise accommodates the coenzyme A portion of the natural substrate. To facilitate binding interaction between an inhibitor and an HMG-CoA reductase enzyme, the side chains (preferably) exits in the stereochemistry of the structures illustrated in FIG. 3.

In some other embodiments, the lipophilic moiety of compounds of formula I or II is varied and tested for MAP kinase and/or HMG-CoA reductase modulatory activity. In some embodiments, the lipophilic moiety is randomly selected. In some embodiments, the lipophilic moiety is selected on the basis of structural diversity or similarity to a MAP kinase modulator, preferably to a lipophilic moiety of a MAP kinase activator. In some embodiments, the lipophilic moiety analog is selected on the basis of structural compatibility with binding to a MAP kinase, for example, using pharmacophore modeling to indicate binding compatibility. In some embodiments, the lipophilic moiety is selected on the basis of structural diversity or similarity to an HMG-CoA reductase inhibitor or on the basis of structural compatibility with binding to an HMG-CoA reductase, for example, using pharmacophore modeling to indicate binding compatibility. Selected lipophilic moieties can be appended with an A-lactone, -lactam, -sulfonyl ester, -sulfuryl ester, -sulfonyl amide, -sulfuryl amide, -dihyropyran and/or -dihydrothiopyran moieties as defined in Formula I, and then tested for activation and/or inhibition of one or more MAP kinase(s) and/or inhibition of an HMG-CoA reductase.

Compounds can be designed and tested entirely using computational methods or a portion of such designing and testing can be done computationally and the remainder done with wet lab techniques.

Testing involves evaluation of the designed compounds for modulatory activity towards one or more MAP kinase(s) and/or HMG-CoA reductase. In some embodiments, the collection of designed analogs may be evaluated by computational methods to predict their activity in modulating one or more MAP kinase(s) and/or HMG-CoA reductase, without physically synthesizing the compounds. Such computational methods may also be used to predict other properties of the compounds, such as solubility, membrane penetrability, metabolism and toxicity.

In some embodiments, testing involves synthesizing the designed compounds and evaluating their activity in modulating one or more MAP kinase(s) and/or HMG-CoA reductase in one or more biological assays via wet lab techniques. Known methods for the synthesis of inhibitors of HMG-CoA reductase and MAP kinases can be adapted to prepare the designed analogs, e.g., in either the δ-lactone or the hydroxy carboxylic acid form, as well as in carboxylate (salt) form.

The modulatory activity of the synthesized compound can then be evaluated by a biological assay, which directly or indirectly reflects the activation and/or inhibition of a MAP kinase, and/or the inhibition of HMG-CoA reductase. For example, known biological assays may be used that determine potency and/or efficacy of the candidate compound in activating and/or inhibiting different p38 MAP kinase isoforms, preferably human p38 MAP kinase isoforms.

Representative biological assays include, but are not limited to, (1) cell-free studies of MAP kinase activity, e.g., using recombinant p38 MAP kinase isoforms or p38 isoforms isolated from tissue samples; (2) studies of the phosphrylation state of p38 MAP kinases, their direct substrates, and their downstream substrates, e.g., in whole cells using antibody-based detection methods; (3) whole-cell studies of stimulation and/or inhibition of inflammatory responses involving the sequelae of activation of p38 MAP kinase pathways (such as cytokine production and/or release upon challenge by agents, including lipopolysaccharide (LPS), increased glucose uptake and/or increased cholesterol efflux); (4) in vivo models of efficacy against MAP kinase-related conditions, such as immunostimulation against infectious agents and/or cancerous cells, or efficacy in treating lipid disorders such as hypercholesterolemia and their sequelae including atherosclerosis, myocardial infarction and occlusive stroke; as well as (5) cell-free studies of HMG-CoA reductase inhibition, using recombinant HMG-CoA reductase and/or HMG-CoA reductase isolated from natural sources; (6) whole-cell studies of cholesterol synthesis and LDL receptor expression; (7) whole cell studies of terpene and sterol biosynthesis; (8) in vivo models of efficacy in treating HMG-CoA reductase-related conditions, such as hypercholesterolemia, lipid disorders such as hyperlipidemia, and atherogenesis and its sequelae, including atherosclerosis, other vascular inflammatory conditions, myocardial infarction, ischemic stroke, occlusive stroke, peripheral occulsive disease, and other peripheral vascular diseases. Such methods are known in the art and/or may be adapted by those skilled in the art.

With respect to in vitro assays, the ability of a candidate compound to modulate one or more MAP kinase and/or HMG-CoA reductase activity can be evaluated by contacting the compound with an assay mixture for measuring activity of one or more MAP kinase(s) and/or HMG-CoA reductase, and determining the activity of the enzyme(s) in the presence and absence of the compound. An increase in activity of a MAP kinase in the presence as opposed to the absence of the compound indicates a MAP kinase activator, at the concentration used. A decrease in activity of a MAP kinase in the presence as opposed to the absence of the compound indicates a MAP kinase inhibitor, at the concentration used. A decrease in the activity of HMG-CoA reductase in the presence as opposed to the absence of the compound indicates an HMG-CoA reductase inhibitor, at the concentration used. MAP kinases and HMG-CoA reductase are known and commercially available, facilitating simple in vitro assays for modulatory activity.

An example of a cell-free MAP kinase assay involves that described in Clerk et al., FEBS Lett. 426:93-96 (1998), incorporated herein by reference. Briefly, serum can be withdrawn from neonatal rats and myocytes exposed to sorbitol (about 30 min) in the absence or presence of about 10 μM or less of a candidate compound. SAPKs/JNKs can be separated by FPLC on a Mono Q HR5/5 column where the MAP kinases are eluted using about a 30 ml linear NaCl gradient (about 0 to about 0.5 M NaCl). They can be assayed by the direct method with myelin basic protein (MBP) or about 0.5 mg/ml glutathione S-transferase-GST-c-Jun(1-135) as substrate, where the assay mix contains about 0.1% (v/v) dimethyl sulphoxide or about 10 μM of a candidate compound (final concentrations). Samples of fractions can be taken for in-gel kinase assays. Fractions may be pooled and concentrated by ultra-filtration and prepared for immunoblot analysis. For MAP-KAPK2, proteins can be applied to a Mono S HR5/5 column and MAP-KAPK2 purified and assayed. GST-c-Jun(1-135) can be used to “pull down” total SAPKs/JNKs from myocyte extracts. Pellets can be washed in kinase assay buffer (for example, about 20 mM HEPES pH about 7.7, about 2.5 mM MgCl₂, about 0.1 mM EDTA, about 20 mM β-glycerophosphate) containing the final concentrations of a candidate compound. The pellets can be re-suspended in about 15 μl kinase assay buffer containing twice the final concentrations of a candidate compound and phosphorylation can be initiated with about 15 μl of kinase assay buffer containing about 10 μM ATP and about 1 μCi [γ-³²P]ATP. JNK1 isoforms can be immunoprecipitated from myocyte extracts using antibodies. The pellets can be washed in kinase assay buffer containing the final concentrations of a candidate compound. GST-c-Jun(1-135) in about 15 μl kinase assay buffer containing twice the final concentrations of a candidate compound can be added and phosphorylation initiated with about 15 μl of kinase assay buffer containing about 20 μM ATP and about 2 μCi [γ-³²P]ATP. Example 5, below, provides further details of a human p38α MAP kinase inhibition assay, as results using a number of candidate compounds.

An example of a cell-free HMG-CoA reductase assay involves radiometric procedures described in Shum et al., Ther. Drug Monit., 20:41-49 (1998), incorporated herein by reference. Briefly, about 150 μg/mL of HMG-CoA reductase can be incubated with a candidate compound, together with about 12 μM [¹⁴C]HMG-CoA and about 200 μM NADPH in about 200 μL 0.2M phosphate buffer (pH about 7.2) for about 0.5 h at about 37° C. The [¹⁴C]mevalonate that forms can be converted under acidic conditions to [¹⁴C]mevalonolactone and separated from un-reacted substrate, for example, by ion-exchange chromatography, and then quantified, for example, by liquid scintillation counting.

An example of a whole cell assay of activation or inhibition of inflammatory responses involves evaluating murine thymic T cell proliferation and IL-2 production or gene expression in the presence and absence of a candidate compound. Methods for measuring T cell proliferation and IL-2 production are standard, well known techniques in the art. Other examples of whole cell assays for inflammation are also known in the art, for example, as described in Welker et al., Int. Arch. Allergy & Immunol. 109:110-115 (1996); Schindler et al., Blood 75:40 (1990); and Golenbock et al., J. Biol. Chem. 266:19490 (1991), incorporated herein by reference. Example 6, provided below, further details a whole-cell anti-inflammation assay useful in certain rational design embodiments of the present invention. Example 7, provided below, further details a whole-cell LPS-stimulated TNF-α release assay also useful in certain rational design embodiments of the present invention.

Animal models used to reflect inflammatory or immune responses can be utilized to evaluate MAP kinase modulatory activity in vivo. Exemplary animal models include, but are not limited to, release of inflammatory mediators in response to LPS administration to mice or rats; the mouse acute irritant model; inbred NC/Nga mice, which develop chronic relapsing skin inflammation when reared under non-pathogen-free conditions; Balb/c mice, which develop dermatitis when injected with Shistosomajaponica glutathione-S-transferase; mice sensitized by repetitive epicutaneous exposure to ovalbumin antigen that model atopic dermatitis; and dextran sulfate sodium, trinitrobenzene sulfonic acid, and oxazolone-induced colitis, which model inflammatory bowel disease. See also, Nagai et al. J. Pharmacol. Exp. Therapeutics 288:43-50 (1999); Boismenu et al. J. Leukoc. Biol., 67:267-278 (2000); and Blumberg et al., Curr. Opinion in Immunol., 11:648-656 (1999). Further, Example 8 below provides more details of a topical inflammation animal model useful in certain rational design embodiments of the present invention.

In some preferred embodiments, the activity or potency of a compound of formula VIII is similar towards one or more MAP kinase(s) and HMG-CoA reductase, preferably as measured by whole cell and/or in vivo assays of AC 50, IC50 or ED50 values, as described in more detail above. In a highly preferred embodiment, the closed, e.g., lactone, form of a compound (Formula I) is the more potent form towards one or more MAP kinase(s), and the open, e.g., hydroxy carboxylic acid or carboxylate (salt), form (Formula II) is the more potent form against HMG-CoA reductase.

All publications, patents, and patent applications mentioned in this specification are herein incorporated by reference to the same extent as if each individual publication, patent, or patent application was specifically and individually indicated to be incorporated by reference.

### EXAMPLES

The following examples are intended to illustrate details of the invention, without thereby limiting it in any manner.

### Example 1

### Synthesis of Atorvastatin Lactone

5.0 g (8.6 mmole) of atorvastatin calcium was dissolved in 300 mL ethyl acetate and washed with 300 mL 10% (w/v) aqueous sodium hydrogen sulfate solution (pH 3). The organic phase was dried over anhydrous magnesium sulfate, filtered and the solvent removed under reduced pressure to afford 2.85 g (5.11 mmole) of atorvastatin acid. This material was dissolved in 300 mL anhydrous toluene and heated at 60° C. for 40 hours, at which time analytical thin-layer chromatography using 4:1 methylene chloride:acetone eluent indicated near-complete conversion of the starting acid to a less polar product. The toluene was removed under reduced pressure and the reaction mixture was fractionated on 300 cc of silica gel using 4:1 methylene chloride: acetone eluent to afford, after combining, concentrating and drying of the appropriate fractions, 2.14 g (3.96 mmol, 46% overall) of atorvastatin lactone as a white foam. The 400 MHz ¹H nuclear magnetic resonance (NMR) spectrum and the electrospray mass spectrum (ES-MS) were consistent with the lactone product. ¹H NMR (Me₂SO-d₆) δ 9.80 (s, 1H), 7.49 (d, 2H), 7.25-7.15 (m, 6H), 7.05 (s, 4H), 6.99 (t, 2H), 5.15 (d, 1H), 4.46 (br s, 1H), 4.02 (s, 1H), 3.97 (m, 1H), 3.89 (m, 1H), 3.21 (q, 1H), 2.55 (dd, 1H), 2.32 (dd, 1H), 1.74 (br s, 2H), 1.6 (m, 2H), 1.36 (d, 6H). ES-MS: obsvd. m/z 541 ([MH]⁺).

### Example 2

### Synthesis of Fluvastatin Lactone

7.0 g (16 mmole) of fluvastatin sodium was dissolved in 300 mL ethyl acetate and washed with 300 mL 10% (w/v) aqueous sodium hydrogen sulfate solution (pH 3). The organic phase was dried over anhydrous magnesium sulfate, filtered and the solvent removed under reduced pressure to afford 5.76 g (14.0 mmole) of fluvastatin acid. This material was dissolved in 300 mL anhydrous toluene and stirred at room temperature for 7 days, at which time analytical thin-layer chromatography using 5:1 methylene chloride:acetone eluent indicated approximately 30% conversion of the starting acid to a less polar product. The toluene was removed under reduced pressure and the reaction mixture was fractionated on 400 cc of silica gel using 5:1 methylene chloride: acetone eluent to afford, after combining, concentrating and drying of the appropriate fractions, 2.02 g (5.14 mmol, 32% overall) of fluvastatin lactone as a white foam. The 400 MHz ¹H nuclear magnetic resonance (NMR) spectrum and the electrospray mass spectrum (ES-MS) were consistent with the lactone product. ¹H NMR (Me₂SO-d₆) δ 7.7 (d, 1H), 7.4 (br s, 3H), 7.3 (m, 2H), 7.2 (t, 1H), 7.1 (t, 1H), 6.8 (t, 1H), 5.7 (dd, 1H), 5.3 (s, 1H), 5.2 (m, 1H), 4.9 (m, 1H), 4.1 (br s, 1H), 2.7 (dd, 1H), 2.4 (d, 1H), 1.8 (d, 1H), 1.7 (t, 1H), 1.57 (d, 6H). ES-MS: obsvd. m/z 394 ([MH]⁺).

Also obtained was a slightly more polar product which ¹H NMR indicated to be the threo-epimer of fluvastatin lactone formed by inversion at the C5 lactone ester center, in accord with the findings of Stokker et al., Heterocycles 26, 157 (1997). This isomer was obtained in an amount of 0.173 g (0.440 mmole, 2.8% overall). ¹H NMR (Me₂SO-d₆) δ 7.7 (d, 1H), 7.42 (m, 3H), 7.3 (t, 2H), 7.2 (t, 1H), 7.05 (t, 1H), 6.8 (t, 1H), 5.7 (dd, 1H), 5.2 (s, 1H), 4.9 (m, 1H), 4.9 (m, 2H), 4.1 (m, 1H), 2.8 (dd, 1H), 2.7 (dd, 1H), 2.3 (dd, 1H), 2.2 (m, 1H), 1.6 (d, 6H).

Using the procedures outlined in Examples 1 and 2, other compounds of Formula II are converted to compounds of Formula. I.

### Example 3

### Synthesis of Simvastatin Sodium

1.43 g (3.42 mmole) of simvastatin lactone was dissolved in 10 mL acetonitrile and treated with water (5 mL) and sodium hydroxide (151 mg, 3.78 mmole). The reaction was stirred at room temperature for 3 days, at which time analytical thin-layer chromatography using 4:1 methylene chloride:acetone eluent indicated essentially complete conversion of the starting lactone to a more polar product. The reaction mixture was then diluted to 50 mL with 1:1 acetonitrile:water, frozen and lyophilized to afford 1.22 g (2.66 mmole, 77.8%) of simvastatin sodium as a fluffy white solid. The 400 MHz ¹H nuclear magnetic resonance (NMR) spectrum was consistent with the sodium carboxylate product. ¹H NMR (Me₂SO-d₆) δ 7.6 (br s, 1H), 5.95 (d, 1H), 5.8 (m, 1H), 5.5 (s, 1H), 5.1 (s, 1H), 4.6 (s, 1H), 3.7 (br s, 1H), 3.5 (br s, 1H), 2.3 (m, 2H), 2.2 (d, 1H), 2.0 (m, 2H), 1.8 (m, 2H), 1.6-1.3 (5H), 1.2 (br s, 3H), 1.0 (9H), 0.80 (m, 3H), 0.75 (m, 3H).

Using the procedure outlined in Example 3, other compounds of Formula I are converted to compounds of Formula II.

### Example 4

### Pharmaceutical Compositions Comprising Compounds of Formula I, II, III or IV for Local/Regional Applications

### Example 4a

### Cerivastatin Lactone Ointment for Ocular Use

| | | |
|---|---|---|
| Cerivastatin lactone | 2.0 | g |
| White petrolatum | 97.5 | g |
| Chlorobutanol | 0.50 | g |

### Example 4b

### Atorvastatin Lactone Skin Ointment in Petrolatum USP Ointment

| | | |
|---|---|---|
| Atorvastatin lactone | 2.0 | g |
| Petrolatum USP | 98 | g |

### Example 4c

### Fluvastatin Lactone Skin Ointment in Hydrophilic Petrolatum USP

| | |
|---|---|
| Fluvastatin lactone | 3.0 g |
| Cholesterol | 3.0 g |
| Stearyl alcohol | 3.0 g |
| White wax | 8.0 g |
| White petrolatum | 86 g |

### Example 4d

### Cerivastatin Lactone Skin Ointment in Hydrophilic Ointment USP

| | | |
|---|---|---|
| Cerivastatin lactone | 0.5 | g |
| Methylparaben | 0.025 | g |
| Propylparaben | 0.015 | g |
| Sodium lauryl sulfate | 1.0 | g |
| Propylene glycol | 12 | g |
| Stearyl alcohol | 25 | g |
| White petrolatum | 25 | g |
| Purified water | 37 | g |

### Example 4e

### Pitavastatin Lactone Skin Ointment in Polyethylene Glycol Ointment NF

| | |
|---|---|
| Pitavastatin lactone | 2.0 g |
| Polyethylene glycol 3350 | 40 g |
| Polyethylene glycol 400 | 60 g |

### Example 4f

### Pitavastatin Lactone Retention Enema

| | | |
|---|---|---|
| Pitavastatin lactone | 0.010 | g |
| Sodium carboxymethyl | 1.0 | g |
| cellulose USP | | |
| Distilled water | 100 | mL |

### Example 4g

### Fluvastatin Lactone Rectal Suppository

| | | |
|---|---|---|
| Fluvastatin lactone | 0.10 | g |
| Theobroma oil | 2.0 | cc |

### Example 4h

### Atorvastatin Lactone Rectal Suppository

| | |
|---|---|
| Atorvastatin lactone | 0.10 g |
| Polyethylene glycol 1000 | 1.5 g |
| Polyethylene glycol 4000 | 0.5 g |

### Example 4i

### Pitavastatin Lactone Dry Powder Aerosol Formulation

| | |
|---|---|
| Pitavastatin lactone | 0.004 g |
| Lactose | 0.0085 g |
| (The mixture is micronized to mass | |
| median particle size between 3-6 μm) | |

### Example 4k

### Fluvastatin Lactone Metered-Dose Aerosol Formulation

| | | |
|---|---|---|
| Fluvastatin lactone | 0.080 | g |
| (Micronized to mass median | | |
| particle size between 3-6 μm) | | |
| Ethanol USP | 0.20 | g |
| Dichlorodifluoromethane | 19.72 | g |
| (Propellant) | | |

### Example 5

### Human p38α MAP Kinase Modulation Assay

In vitro cell-free p38α MAP kinase modulation assays were conducted by the method as described in Clerk et al., supra, (1998). Briefly, human recombinant p38α protein kinase expressed in E. Coli (UBI #14-251) was used. Myelin basic protein (MBP, UBI #13-110) was employed as substrate, and microtiter plate wells were coated with MBP (0.01 mg/ml) overnight at 4° C. Candidate compound and/or vehicle was preincubated with 0.075 μg/mL enzyme in modified HEPES buffer pH 7.4 at 25° C. for 15 minutes. The reaction was initiated by addition of 100 μM ATP and allowed to proceed for another 60 minutes. The reaction was terminated by aspirating the solution. Phosphorylated MBP was detected by incubation with a mouse monoclonal IgG2a anti-phosphoMBP antibody. Bound anti-phosphoMBP antibody was quantitated by incubation with a HRP conjugated goat anti-mouse IgG. The protein kinase activity was proportional to the readings of optical density at 405 nm resulting from reaction with an ABTS Microwell Peroxidase Substrate System. Using this method, IC50, AC50 and Max % Act data can be obtained, e.g., providing results illustrated in Table I, as discussed above.

### Example 6

### Whole Cell Inflammation Assay

The procedure as described in Welker et al., supra, (1996) can be followed. That is, peripheral blood mononuclear cells (PBMCs) can be prepared from four different donors by differential centrifugation on Ficoll-Hypaque (Seromed, Berlin, Germany). Two donors (1 and 2) may have seasonal rhino-conjunctivitis, e.g., with positive prick tests to inhalant allergens and elevated serum IgE levels. PBMCs may contain approximately 10% CD14-positive monocytic cells, approximately 90% lymphocytes and approximately <1% granulocytes and platelets.

THP-1 cells are obtained from the ATCC (Rockville, Md., USA; TIB 202) and can be routinely kept in RPMI medium (Gibco, Eggenstein, Germany) with 10% FCS (Seromed) and 50 μM mercaptoethanol (Gibco) added. HL-60 cells (ATCC; No. CCL 240) can be kept in RPMI medium, with 20% FCS, and U-937 cells (ATCC; No. CCL 1593) can be kept in RPMI medium with 10% FCS.

The following glucocorticoids are dissolved in DMSO: Methylprednisolone aceponate (MPA), methylprednisolone-17-propionate (MPP), prednicarbate (PC) and betamethasone valerate (BMV) (Schering, Berlin, Germany). The stock solutions are diluted with medium to <0.1% DMSO before use to avoid toxic effects on the cells.

All cells (10⁶/ml) can be kept in 24-well polystyrene culture plates and stimulated with lipopolysacharide (LPS; 50 ng/ml; Sigma, St. Louis, Mo., USA) for 24 h at 37° C. in RPMI medium (Gibco) without serum, alone or with 10−5-⁻⁸ M GC added.

THP-1, HL-60 and U-937 cells can also be stimulated with a combination of phorbol myristate acetate (PMA; 25 ng/ml) and the calcium ionophore A23187 (Ion; 2x⁻⁷ M; both from Sigma). In pre-incubation experiments, cells are cultured for 1 h with the different GCs (10⁻⁶ M) before addition of the stimulus. As controls, cells are cultured with medium only, without stimulus or GCs and with 0.1% DMSO. After incubation, cells are centrifuged, and the culture supernatants frozen at −20° C. until analysis.

Cytokines (IL-1β, 1L-8 and TNF-α) in cell supernatants can be quantified by ELISA (Quantikine, Biermann, Bad Nauheim, Germany), and data can be expressed as means of two values calculated for 10⁶ viable cells. Data of duplicate measurements may fluctuate within a very narrow margin (<5%). All experiments can be repeated three (cell lines) or four (PBMC) times. 5×10⁷ THP-1 cells stimulated for 24 h with or without PMA/A23187 and with or without 10⁻⁶ M MPA can be lysed with 3 M lithium chloride and 6 M urea, centrifuged at 20,000 rpm for 60 min, and RNA extracted in phenol-chloroform.

8 μg total RNA per lane can be electrophorased and transferred to nitrocellulose membranes (NEN Research, Boston, Mass., USA) by standard techniques. For Northern blot hybridization, HindIII/Bam-HI DNA fragments of TNF-α (680 bp) can be used. The fragments can be nick translated using ³²P-labeled dCTP (NEN Research) and a random primer labeling kit (Boehringer, Mannheim, Germany). Hybridization can be carried out in SSC (NaCl/sodium citrate) (Sigma) buffer containing 50% formamide (Sigma) and 10% dextran sulfate (Sigma) over-night at 42° C., according to standard procedures. On the following day, nitrocellulose membranes can be washed twice in 2×SSC buffer containing 0.1% sodium dodecyl sulfate (SDS; Sigma) for 15 min at 42° C. and twice in 0.2×SSC containing 0.1 SDS at 50° C. After drying, the blot can be exposed to an X-ray film (Kodak, Rochester, Mass., USA) for up to 7 days.

Statistical significance may be calculated with the two-tailed t-test. The IC₅₀ data (inhibitory constants) may be calculated as the GC concentration that cause 50% inhibition of cytokine release, using a computer-assisted program (SPSS, Microsoft). With respect to candidate activators, AC₅₀ data may be calculated as the concentration of candidate compound that causes 50% increase in cytokine release, using a computer-assisted program (SPSS, Microsoft) and Max % Act data may be calculated as the maximum increase in cytokine release observed in the presence of the candidate compound at a particular concentration.

### Example 7

### Whole Cell LPS-Stimulated TNF-α Release Assay

The procedure as described in Welker et al., supra, (1996) can be followed. Briefly, a candidate compound and/or vehicle can be preincubated with human peripheral blood mononuclear leukocytes (PBML, 5×10⁵/ml) cells in AIM-V medium pH 7.4 for 2 hours. Lipopolysaccharide (LPS, 25 ng/ml) can be added to stimulate the cells, which can be incubated overnight at 37° C. TNF-α cytokine levels in the conditioned medium can then be quantitated using a sandwich ELISA kit.

### Example 8

### Topical Inflammation Model

Groups of 5 BALB/c male mice weighing 22±2 g can be sensitized by application of oxazolone (100 μL, 1.5% v/v in acetone) to the shaved abdominal surface. Seven days after sensitization, a candidate compound (0.1-5 mg in 20 μL acetone, methanol or ethanol vehicle) or vehicle alone (20 μL) can be applied topically to the anterior and posterior surfaces of the right ear 30 minutes before and 15 minutes after oxazolone (1% v/v, 25 μL/ear) challenge applied in the same manner to the right ear. Left ears can be untreated. The thickness of both ears of each animal can be measured with a Dyer model micrometer gauge 24 hours after oxazolone challenge, and the net increase in thickness of right ears versus left ears can be calculated for each animal. Percent inhibition can be calculated according to the formula: [(Iv−It)/Iv]×100, where Iv and It respectively refer to the average net increase in right ear thickness (mm) for vehicle and candidate compound treated mice. Percent activation can be calculated according to the formula: [(It−Iv)/Iv]×100.

The above examples are in no way intended to limit the scope of the instant invention. Further, it can be appreciated to one of ordinary skill in the art that many changes and modifications can be made thereto without departing from the spirit or scope of the appended claims, and such changes and modifications are contemplated within the scope of the instant invention.

What is claimed is:

1. A method of activating a MAP kinase comprising administering an effective amount of a composition comprising a statin lactone.

2. The method as recited in claim 1 wherein said activation occurs in a cell other than a brain cell.

3. The method as recited in claim 1 wherein said activating occurs by direct activation.

4. The method as recited in claim 1 wherein said activating does not occur via a growth factor.

5. The method as recited in claim 1 wherein said activating is not reversed by addition of at least one compound selected from farnesyl pyrophosphate, geranylgeranyl pyrophosphate and mevalonte.

6. The method as recited in claim 1 wherein said activating is not reversed by addition of a downstream product of mevalonate.

7. The method as recited in claim 1 wherein said MAP kinase is a p38 MAP kinase.

8. The method as recited in claim 1 wherein said MAP kinase is a p38α MAP kinase.

9. The method as recited in claim 8 wherein said statin lactone is simvastatin lactone.

10. The method as recited in claim 8 wherein said statin lactone is cerivastatin lactone.

11. The method as recited in claim 8 wherein said statin lactone is fluvastatin lactone.

12. The method as recited in claim 8 wherein said statin lactone is lovastatin lactone.

13. The method as recited in claim 8 wherein said statin lactone is mevastatin lactone.

14. The method as recited in claim 8 wherein said statin lactone is not atorvastatin lactone, rosuvastatin lactone, nor pitavastatin lactone.

15. The method as recited in claim 1 wherein said MAP kinase is a p38β MAP kinase.

16. The method as recited in claim 15 wherein said statin lactone is simvastatin lactone.

17. The method as recited in claim 15 wherein said statin lactone is cerivastatin lactone.

18. The method as recited in claim 15 wherein said statin lactone is fluvastatin lactone.

19. The method as recited in claim 15 wherein said statin lactone is atorvastatin lactone.

20. The method as recited in claim 15 wherein said statin lactone is not rosuvastatin lactone.

21. The method as recited in claim 1 wherein said MAP kinase is a p38γ MAP kinase.

22. The method as recited in claim 21 wherein said statin lactone is simvastatin lactone.

23. The method as recited in claim 21 wherein said statin lactone is cerivastatin lactone.

24. The method as recited in claim 21 wherein said statin lactone is rosuvastatin lactone.

25. The method as recited in claim 21 wherein said statin lactone is atorvastatin lactone.

26. The method as recited in claim 21 wherein said statin lactone is pitavastatin lactone.

27. The method as recited in claim 21 wherein said stain lactone is not fluvastatin lactone.

28. The method as recited in claim 1 wherein said MAP kinase is a p38δ MAP kinase.

29. The method as recited in claim 28 wherein said statin lactone is simvastatin lactone.

30. The method as recited in claim 28 wherein said statin lactone is cerivastatin lactone.

31. The method as recited in claim 28 wherein said statin lactone is rosuvastatin lactone.

32. The method as recited in claim 28 wherein said statin lactone is atorvastatin lactone.

33. The method as recited in claim 28 wherein said statin lactone is fluvastatin lactone.

34. The method as recited in claim 1 wherein said composition activates at least two MAP kinases.

35. The method as recited in claim 34 wherein said at least two MAP kinases are selected from a p38α MAP kinase, a p38β MAP kinase, a p38γ MAP kinase, a p38δ MAP kinase, and a p42 MAP kinase.

36. The method as recited in claim 34 wherein said statin lactone is at least one lactone selected from simvastatin lactone, cerivastatin lactone, fluvastatin lactone, rosuvastatin lactone, and atorvastatin lactone.

37. The method as recited in claim 1 wherein said MAP kinase is a JNK.

38. The method as recited in claim 37 wherein said activating facilitates a Fas apoptotic pathway.
